(19) United States
(12) Patent Application Publication
(10) Pub. No.: US 2002/0026658 A1
(43) Pub. Date: Feb. 28, 2002

(54) Genes encoding sinapoylglucose: malate sinapoyltransferase and methods of use

(76) Inventor: Clinton C.S. Chapple, West Lafayette, IN (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/901,252

(22) Filed: Jul. 9, 2001

Related U.S. Application Data
(60) Provisional application No. 60/216,593, filed on Jul. 7, 2000.

(51) Int. Cl. A01H 5/00; C12N 15/82; C12N 15/29; C12N 15/62

(52) U.S. Cl. 800/281; 800/278; 536/23.6; 800/302; 435/69.8

(57) ABSTRACT

A gene has been isolated from Arabidopsis encoding sinapoylglucose:malate sinapoyltransferase (SMT). SMT is responsible for the substitution of a glucose moiety on sinapoylglucose with a malate moiety to form sinapoylmalate in plant vacuoles. The enzyme is useful the manipulation of plant secondary metabolism.

This application claims benefit under 35 U.S.C. § 119(e) of Provisional Patent Application Ser. No. 60/216,593, filed Jul. 7, 2000, incorporated herein by reference.

### BACKGROUND OF THE INVENTION

This invention is in the field of plant molecular biology and relates to the utilization of isolated nucleotide sequences to genetically engineer plants, and/or microorganisms. More particularly, the invention relates in certain preferred aspects to novel nucleotide sequences and uses thereof, including their use in DNA constructs for transforming plants and microorganisms. More particularly, the invention pertains to isolated nucleic acid fragments encoding plant sinapoylglucose:malate sinapoyltransferase (SMT) and its use in the manipulation of plant metabolism.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Plants produce thousands of unique molecules that are collectively referred to as secondary metabolites. Even within the angiosperms, many of these compounds are unique to specific taxa, indicating that the pathways that produce them may have evolved within the last 100,000 years. A central question in the study of plant secondary metabolism concerns how the catalytic diversity of plant secondary metabolism has arisen. Another important area of investigation is the identification of the classes of genes and proteins that have been co-opted, presumably from their ancestral roles in primary metabolism, to serve as catalysts in the synthesis of secondary metabolites.

In Arabidopsis, the phenylpropanoid pathway leads to the production of sinapic acid esters, a group of fluorescent UV-protective secondary metabolites derived from phenylalanine. These compounds are dispensable under laboratory conditions, and thus provide targets for the genetic dissection of phenylpropanoid metabolism. The analysis of these compounds is facilitated by their blue fluorescence under UV light both in vivo, and following TLC analysis (Chapple, et al., 1992; Ruegger, et al., 1999). Arabidopsis and some other members of the Brassicaceae accumulate three major sinapic acid esters. In the biosynthetic pathway leading to these compounds, sinapoylglucose is the immediate precursor of sinapoylcholine and sinapoylmalate, which are accumulated in seeds and leaves, respectively. 1-O-Sinapoylglucose is a β-acetal ester that has a high free energy of hydrolysis (Mock and Strack, 1993). It provides the necessary free energy for the transacylation reaction catalyzed by sinapoylglucose:malate sinapoyltransferase (SMT; EC 2.3.1.92) (Strack, 1982), which generates sinapoylmalate in vegetative tissues (Sharma and Strack, 1985). During seed maturation, sinapoylglucose is instead converted to sinapoylcholine by sinapoylglucose:choline sinapoyltransferase (SCT; EC 2.3.1.91) (Strack, et al., 1983). Despite the detailed biochemical understanding of this pathway, none of the genes involved has been cloned, and relatively little is known about the regulation of the pathway. Such information would be useful in designing new methods for the manipulation of plant metabolism.

The problem to be solved therefore is to identify the genes encoding SMT in order to provide a new method for altering plant metabolism, specifically plant secondary metabolism, and most specifically, sinapate ester metabolism. Applicants have solved the stated problem by isolating and sequencing the gene encoding SMT (known as SNG1), by expressing SMT in transformed plants and microorganisms, and by demonstrating that the SNG1 gene product indeed catalyzes the transesterification of sinapoylglucose to sinapoylmalate in vivo and in vitro. Unexpectedly the SNG1 gene and the encoded SMT gene product demonstrate high sequence homology to a class of proteins known as serine carboxypeptidases.

Serine carboxypeptidases have been identified in a wide array of organisms. They catalyze the hydrolysis of the C-terminal peptide bond in proteins or peptides and are usually thought of as being involved in protein degradation and processing. The best studied of these is serine carboxypeptidase Y from Saccharomyces cerevisiae, a vacuolar protein that is initially synthesized as a preproenzyme. This enzyme has been used extensively in studies of protein transport, targeting and processing (for examples, see Valls, et al., 1990; Ramos, et al., 1994; Ramos and Winther, 1996). Alkylation by suicide inhibitors and subsequent mutagenesis experiments have identified the active site serine and histidine residues (Hayashi, et al., 1973; Hayashi, et al., 1975; Bech and Breddam, 1989), and crystallization of the enzyme has permitted the identification of the other amino acid residues that make up the substrate binding pocket (Endrizzi, et al., 1994).

In plants, serine carboxypeptidases and proteins that share amino acid sequence homology with them, also referred to as serine carboxypeptidase-like (hereafter “SCPL”) proteins, have been isolated from a number of species, and SCPL genes have been identified in EST and genomic sequencing projects. The proteins from wheat and barley have been particularly well studied because of their inferred role in mobilization of seed storage reserves (Baulcombe, et al., 1987; Doan and Fincher, 1988; Degan, et al., 1994), and the homodimeric wheat serine carboxypeptidase II has been crystallized (Liao and Remington, 1990; Liao, et al., 1992). SCPL proteins have also been purified and characterized from cauliflower, rice, and tomato (Doi, et al., 1980; Kim and Hayashi, 1983; Mehta and Mattoo, 1996; Mehta, et al., 1996; Walker-Simmons and Ryan, 1980). SCPL genes have been isolated from Arabidopsis, pea and rice by their homology to SCPL cDNAs from wheat and barley (Bradley, 1992; Washio and Ishikawa, 1994; Jones, et al., 1996). SCPL enzymes also play a role in herbicide metabolism where an SCPL protein has been shown to catalyze the first step in the catabolism of an alachlor glutathione S-conjugate by removing the terminal glycine residue of the glutathione moiety (Wolf, et al., 1996). Based upon these and other studies, SCPL enzymes have been suggested to have functions ranging from protein turnover and C-terminal processing to roles in wound responses and xenobiotic metabolism.

Although plant SCPL enzymes and genes have been the subject of numerous publications, their natural substrates are largely unknown. Virtually all SCPL enzymes have been purified from plants based upon their ability to degrade artificial peptide substrates. In many cases, their role in proteolysis has been implied or assumed because the enzymes have been isolated from tissues actively engaged in protein turnover, and show little apparent substrate specificity. While some of these enzymes may be carboxypeptidases, no genetic proof has demonstrated their in vivo function.

### SUMMARY OF THE INVENTION

The present invention provides nucleotide sequences as set forth herein relating to the expression of active sinapoylglucose:malate sinapoyltransferase. Also provided are vectors, expression cassettes and other DNA constructs including such sequences.

Additionally the invention provides transgenic organisms comprising a gene encoding a functional sinapoylglucose:malate sinapoyltransferase, where the transgenic organisms are selected from the group consisting of bacteria, filamentous fungi and plants. In a preferred embodiment the invention provides a method of altering the levels of sinapoylmalate biosynthetic enzymes in a plant comprising: a) transforming a plant with a nucleic acid molecule encoding a polypeptide sequence selected from the group consisting of SEQ ID NO:2, SEQ ID NO:4 and SEQ ID NO:6; and b) growing the transformed plant seed under conditions wherein the polypeptide is expressed.

The present invention also provides methods for the production of active sinapoylglucose:malate sinapoyltransferase. Exemplary methods include (a) introducing into a suitable host cell a nucleic acid molecule selected in accordance with the invention, wherein the nucleic acid molecule is under the control of suitable regulatory elements such that a functional sinapoylglucose:malate sinapoyltransferase is expressed; and (b) recovering the sinapoylglucose:malate sinapoyltransferase produced in step (a). The invention additionally provides methods for the production of sinapoylglucose:malate sinapoyltransferase where the host cells are selected from the group consisting of bacteria, filamentous fungi and plants.

In another aspect, the invention provides a method of obtaining a nucleic acid fragment encoding all or a substantial portion of a plant sinapoylglucose:malate sinapoyltransferase. Exemplary methods include (a) probing a genomic library with the nucleic acid fragment selected a nucleic acid molecule selected in accordance with the invention, (1) identifying a DNA clone that hybridizes with the nucleic acid fragment of step (a); and (c) sequencing the genomic fragment that comprises the clone identified in step (b), wherein the sequenced genomic fragment encodes a functional plant sinapoylglucose:malate sinapoyltransferase.

Similarly the invention provides a method of obtaining a nucleic acid fragment encoding all or a substantial portion or of plant sinapoylglucose:malate sinapoyltransferase comprising: (a) synthesizing at least one oligonucleotide primer corresponding to a portion of the nucleic acid sequence as set forth in SEQ ID NO:1, SEQ ID NO:3 or SEQ ID NO:5 and (b) amplifying an insert present in a cloning vector, cDNA, or genomic DNA using the oligonucleotide primer of step (a); wherein the amplified insert encodes a portion of an amino acid sequence encoding a plant sinapoylglucose:malate sinapoyltransferase.

In an alternate embodiment the invention provides a mutated SNG1 gene encoding a sinapoylglucose:malate sinapoyltransferase having an altered biological activity produced by a method comprising the steps of: (i) digesting a mixture of nucleotide sequences with restriction endonucleases wherein said mixture comprises:

a) A native SNG1 gene;

b) a first population of nucleotide fragments which will hybridize to said native SNG1;

c) a second population of nucleotide fragments which will not hybridize to said native SNG1;

wherein a mixture of restriction fragments are produced; (ii) denaturing said mixture of restriction fragments; (iii) incubating the denatured said mixture of restriction fragments of step (ii) with a polymerase; (iv) repeating steps (ii) and (iii) wherein a mutated SNG1 gene is produced encoding a sinapoylglucose:malate sinapoyltransferase having an altered biological activity.

### BRIEF DESCRIPTION OF THE DRAWINGS AND SEQUENCE DESCRIPTIONS

FIG. 1 illustrates the pathway of sinapate ester biosynthesis including the conversion of sinapoylglucose to sinapoylmalate via sinapoylglucose:malate sinapoyltransferase. The enzymes required for the conversion of phenylalanine to sinapic acid are phenylalanine ammonia4yase (PAL), cinnamate-4-hydroxylase (C4H), p-coumarate-3-hydroxylase (C3H), caffeic acid/5-hydroxyferulic acid O-methyltransferase (OMT) and ferulate-5-hydroxylase (F5H). The enzymes unique to sinapate ester biosynthesis are UDP-glucose:sinapic acid glucosyltransferase (SGT), sinapoylglucose:malate sinapoyltransferase (SMT), sinapoylglucose:choline sinapoyltransferase (SCT) and sinapoylcholinesterase (SCE). The biochemical block in the sng1 mutant is indicated with a horizontal line across the step catalyzed by SMT.

FIG. 2 is an autoradiographic image of an RNA gel blot hybridization analysis of the expression of the putative SNG1 gene.

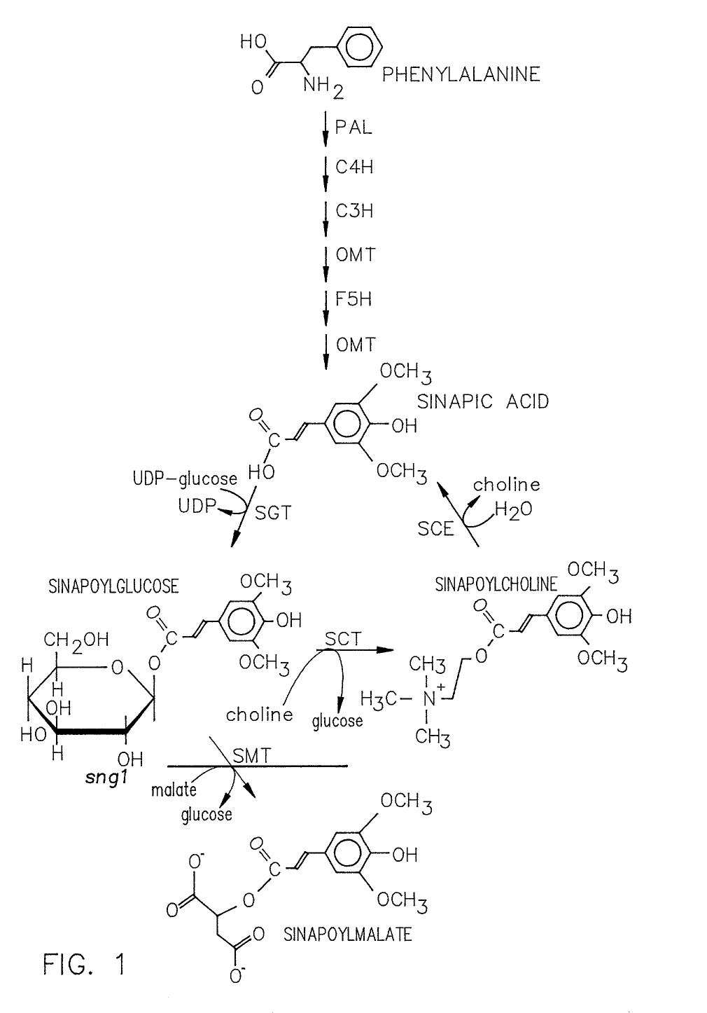

A. Gel blot hybridization of RNA isolated from leaves of wild type and sng1 mutants probed with the 3.9 kb fragment of pBIC20-SNG1. Lane 1, Columbia wild type; lane 2, Wassilewskija wild type; lanes 3-6, sng1-1 through sng1-4.

B. DNA gel blot analysis of fast neutron-induced sng1 alleles. Genomic DNA was prepared from M₂ plants, digested with HindIII, electrophoresed, blotted, and probed with the 10, 3.9, and 4.5 kb HindIII fragments indicated in FIG. 4. Lane 1, Columbia wild type; lanes 2 and 3, two isolates of sng1-5 from a single parental group; lanes 4, 5, and 6, sng1-6, sng1-7, sng1-8.

C. Gel blot hybridization analysis of SNG1 expression in wild-type Arabidopsis. RNA was prepared from various tissues and probed with the 3.9 kb fragment of pBIC20-SNG1. Lane 1, young leaves; lane 2, mature leaves; lane 3, senescent leaves; lane 4, ten day old seedlings; lane 5, stems; lane 6, siliques; lane 7, flowers; lane 8, roots.

FIG. 3 illustrates the region of the Arabidopsis genome surrounding the SNG1 locus. The BAC clone F21P24 was found to include the putative SNG1 gene (SCPL 3) as well as four additional SCPL genes, each of which is indicated with arrows. The sixth SCPL gene is upstream of SCPL1 and is not indicated because it is thought to be a pseudogene. The position of the T-DNA insertion in the sng1-4 allele, and the regions of Arabidopsis genomic DNA carried by the pBIC20-SNG1 and pGA482-SNG1 complementation constructs are indicated.

FIG. 4 is an analysis of sinapate ester content in wild-type, mutant and transgenic lines. Leaf extracts were prepared from Columbia wild type (lane 1), sng1-1 (lane 2) and three sng1-1 transformants carrying the pBIC20-SNG1 transgene (lanes 3 to 5). Extracts were analyzed by TLC on silica gel plates using the mobile phase n-butanol: acetic acid 5:2:3 (v/v/v): water 4:1:1. Sinapoylmalate (sm) and sinapoylglucose (sg) were visualized under 312 nm UV light (o, origin; sf, solvent front).

FIG. 5 is an alignment of the SNG1 gene product with serine carboxypeptidases and serine-carboxypeptidase-like proteins. An alignment of SMT with the yeast carboxypeptidase Y (CPY), wheat carboxypeptidase (CPDW-II), and the hydroxynitrile lyase from Sorghum bicolor (SbHNL) (only an incomplete sequence is available in the database) was prepared using the ClustalW algorithm. Amino acids that are identical in two or more proteins are shaded in black, conservative amino acid substitutions are shaded in gray. Putative active residues in SMT (S-173, D-358, and H-411) are designated with black arrowheads based upon alignment with the carboxypeptidase Y catalytic triad. Dashes denote gaps introduced to optimize the amino acid alignment.

FIG. 6 illustrates an electrophoresis gel comparing the proteins isolated form a soluble and insoluble cell fraction from recombinant E. coli expression SNG1.

A. SDS-PAGE analysis of soluble (A) and insoluble (B) fractions of E. coli harboring pET28A (lanes 1 and 2) and the SNG1 expression vector pET28A-SNG1 (lane 3 and 4) grown in the absence (lanes 1 and 3) or presence (lanes 2 and 4) of 0.8 mM IPTG.

B. The same analysis as given for (A) using insoluble fractions.

FIG. 7 is an analysis of SMT activity in E. coli expressing the SNG1 gene. Enzyme assays and leaf extracts were analyzed by HPLC with UV detection at 335 nm. Assay I contained all assay components, except E. coli protein extract. Assays II to IV contained 100 μg of soluble protein from E. coli harboring pET28A-SNG1; assay II lacked sinapoylglucose (sg); assay III lacked malate. Assay IV contained all assay components. HPLC run V represents a methanolic extract of wild-type Arabidopsis leaves containing sinapoylmalate (sm). Assay VI included all assay components incubated with 100 μg of soluble protein of E. coli harboring the original pET28A vector. All protein extracts were obtained from cultures that had not been induced with IPTG. All assays were incubated at 30° C. for 14 h. The identity of the SMT reaction product was confirmed by LC-MS on a Micromass Quattro Ultima (Micromass, UK) triple quadrupole instrument in negative ion electrospray mode (m/z- for sinapoylmalate 339.15).

The invention can be more fully understood from the following detailed description and the accompanying sequence descriptions which form a part of this application.

The following sequence descriptions and sequences listings attached hereto comply with the rules governing nucleotide and/or amino acid sequence disclosures in patent applications as set forth in 37 C.F.R. §1.821-1.825. The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NO:1 is the genomic nucleotide sequence of the SNG1 gene isolated from Arabidopsis.

SEQ ID NO:2 is the amino acid sequence encoded by the coding region of the sequence of SEQ ID NO:1.

SEQ ID NO:3 is the cDNA sequence for the coding region of the SNG1 gene, including the sequence encoding the signal peptide.

SEQ ID NO:4 is the amino acid sequence of the pre-sinapoylglucose:malate sinapoyltransferase (SMT) enzyme encoded by the SEQ ID NO:3, including the signal peptide.

SEQ ID NO:5 is the nucleotide sequence of the portion of the SNG1 gene encoding a mature SMT isolated from Arabidopsis.

SEQ ID NO:6 is the amino acid sequence of the mature sinapoylglucose:malate sinapoyltransferase encoded by the SNG1 CDNA, not including the sequence encoding the signal peptide, and is the same as amino acids 20-433 of SEQ ID NO:4.

SEQ ID NO:7-8 are primers used for PCR amplification of genomic DNA of the SNG1-4 mutant.

SEQ ID NO:9-10 are primers used for PCR amplification of the SNG1 gene.

SEQ ID NO:11 is the amino acid sequence of the postulated signal peptide found immediately following the predicted cleavage site for SMT.

SEQ ID NO:12-13 are oligonucleotides used to amplify a fragment of the SNG1 cDNA.

SEQ ID NO: 14-17 are comparison of amino acid sequences of SNG1 gene product with serine carboxypeptidases and serine-carboxypeptidase-like proteins. Dashes present on FIG. 5 have been ignored for purposes of preparing the sequence listing.

### DETAILED DESCRIPTION OF THE INVENTION

The present invention relates to DNA constructs that may be integrated into a plant to provide an inventive transformed plant. The present provides a gene (identified herein as “SNG1”) encoding a sinapoylglucose:malate sinapoyltransferase (SMT) enzyme which has the ability to convert sinapoylglucose to sinapoylmalate. The gene can be advantageously used for the recombinant expression of the SMT protein and its activity has been confirmed by in vitro assays.

The SNG1 gene encodes the last enzyme in the sinapoylmalate biosynthetic pathway, converting sinapoylglucose to sinapoylmalate. Considering that phenolic acid glucose esters such as sinapoylglucose are common in plant metabolism, introduction of the SNG1 gene into plants is expected to result in modification of the biosynthesis of glucose ester secondary metabolism. More specifically, modification of SNG1 gene expression is useful for the modification of phenolic acid glucose ester metabolism in plants. Most specifically, modification of SNG1 gene expression is useful for the modification of sinapate ester metabolism in plants.

To isolate the SNG1 gene, a screen of 7600 EMS-mutagenized Arabidopsis plants was conducted. TLC analysis of methanolic leaf extracts, as described in greater detail in the Examples below, identified two allelic mutants that lacked sinapoylmalate, and instead accumulated its biosynthetic precursor, sinapoylglucose (Lorenzen, et al., 1996). Based upon their biochemical phenotype, these mutants were named sng1-1 and sng1-2 (sinapoylglucose accumulator 1). While SMT activity was readily detectable in wild-type leaf extracts, it was undetectable in extracts of mutant leaf tissue.

The phenotype of the sng1 mutant indicates that the SNG1 gene encodes a protein required for SMT activity or expression. This means that the SNG1 gene could encode a transcription factor that activates expression of the SMT gene, a protein that blocks the degradation of SMT, a protein required for the post translational activation of SMT, a protein required for the synthesis of an SMT cofactor or prosthetic group (Schwartz, et al., 1997), or SMT itself.

The present invention relates to the discovery that the SNG1 locus encodes SMT and that SMT exhibits homology with serine carboxypeptidases (EC 3.4.16.1). Considering that serine carboxypeptidases are hydrolases that use proteins or peptides as substrates, the present invention demonstrates a novel activity for enzymes belonging to this class of proteins. The present invention thus provides a gene and gene product having a high degree of homology to serine carboxypeptidase enzymes, but demonstrating the ability to substitute the glucose moiety of the glucose ester of sinapic acid (sinapoylglucose) with a malate molecule. The malate conjugated enzymatic reaction product has been found to be localized in the plant vacuole.

In this disclosure, a number of terms and abbreviations are used. The following definitions are provided.

“Open reading frame” is abbreviated ORF.

“Polymerase chain reaction” is abbreviated PCR.

“SCPL” is the abbreviation for serine carboxypeptidase-like.

“SMT” refers to the enzyme sinapoylglucose:malate sinapoyltransferase

“sng1” refers to the Arabidopsis mutant “sinapoylglucose accumulator 1” which accumulates sinapoylglucose instead of sinapoylmalate, and lacks SMT activity due to a defect in the SMT gene.

“SNG1” refers to the gene locus which encodes the enzyme sinapoylglucose:malate sinapoyltransferase.

“SGT” is the abbreviation for the enzyme UDP-glucose:sinapic acid glucosyltransferase which is responsible for the conversion of sinapic acid to sinapoylglucose.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more usually at least about 80% identity, preferably at least about 90% identity, and more preferably at least about 95% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Lesk, A. M., ed., 1988; Smith, D. W., ed., 1993; Griffin and Griffin, eds., 1994; von Heinje, 1987; and Gribskov and Devereux, eds., 1991). Methods commonly employed to determine identity between two sequences include, but are not limited to those disclosed in Guide to Huge Computers, Martin J. Bishop, ed., Academic Press, San Diego, 1994, and Carillo and Lipman, 1988. Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, et al., 1984), BLASTP, BLASTN, FASTA (Altschul, et al., 1990; Altschul, et al., 1997). The well-known Smith Waterman algorithm may also be used to determine identity.

As an illustration, by a polynucleotide having a nucleotide sequence having at least, for example, 95% “identity” to a reference nucleotide sequence of is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30C, typically in excess of 37C, and preferably in excess of 45C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Ausubel, 1987; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5× SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1× SSC at about 65° C.

As used herein, “substantially similar” refers to nucleic acid molecules wherein changes in one or more nucleotide bases does not result in substitution of one or more amino acids. “Substantially similar” also refers to nucleic acid molecules wherein changes in one or more nucleotide bases results in substitution of one or more amino acids, but do not affect the functional properties of the protein encoded by the DNA sequence. “Substantially similar” also refers to nucleic acid molecules wherein changes in one or more nucleotide bases does not affect the ability of the nucleic acid molecule to mediate alteration of gene expression by antisense or co-suppression technology. “Substantially similar” also refers to modifications of the nucleic acid molecules of the instant invention such as deletion or insertion of one or more nucleotide bases that do not substantially affect the functional properties of the resulting transcript. “Substantially similar” also refers to a polypeptide encoded by such nucleic acid molecules. It is therefore understood that the invention encompasses more than the specific exemplary sequences.

For example, it is well known in the art that alterations in a gene which result in the production of a chemically equivalent amino acid at a given site, but do not effect the functional properties of the encoded protein are common. In this manner, it is known that serine may commonly be substituted with threonine in a polypeptide without eliminating the functionality of the polypeptide. The following sets forth groups of amino acids which are believed to be interchangeable in inventive amino acid sequences at a wide variety of locations without eliminating the functionality thereof:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functional product. The term “functional product” is intended to identify a product that has at least one function in common with the polypeptides described herein. With respect to SMT, a functional product functions to convert sinapoylglucose to sinapoylmalate, even if the conversion is less efficient than conversion by native SMT. Where one is unsure whether a given substitution will eliminate the functionality of the enzyme, this may be determined without undue experimentation using synthesis techniques and screening assays known in the art.

In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, the skilled artisan recognizes that preferred substantially similar sequences encompassed by this invention are those defined by their ability to hybridize, under stringent conditions (0.1× SSC, 0.1% SDS, 65° C. and washed with 2× SSC, 0.1% SDS followed by 0.1× SSC, 0.1% SDS), with the sequences exemplified herein.

Additional preferred substantially similar nucleic acid molecules of the instant invention are those nucleic acid molecules whose DNA sequences have at least 80% identity to the DNA sequence of a nucleic acid molecule reported herein. More preferred nucleic acid molecules have at least 90% identity to the DNA sequence of a nucleic acid molecule reported herein. Most preferred are nucleic acid molecules that have at least 95% identity to the DNA sequence of a nucleic acid molecule reported herein.

Additional preferred substantially similar nucleic acid molecules of the instant invention are those nucleic acid molecules that encode polypeptides whose amino acid sequences have at least 80% identity to the amino acid sequence of a polypeptide reported herein. More preferred polypeptides have at least 90% identity to the amino acid sequence of a polypeptide reported herein. Most preferred are polypeptides that have at least 95% identity to the amino acid sequence of a polypeptide reported herein.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, et al., Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. For preliminary screening for homologous nucleic acids, low stringency hybridization conditions, corresponding to a Tm of 55°, can be used, e.g., 5× SSC, 0.1% SDS, 0.25% milk, and no formamide; or 30% formamide, 5× SSC, 0.5% SDS. Moderate stringency hybridization conditions correspond to a higher Tm, e.g., 40% formamide, with 5× or 6× SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

A “substantial portion” of an amino acid or nucleotide sequence is enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, et al., 1993; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid molecule comprising the primers. Accordingly, a “substantial portion” of a nucleotide sequence comprises enough of the sequence to specifically identify and/or isolate a nucleic acid molecule comprising the sequence. The instant specification teaches partial or complete amino acid and nucleotide sequences encoding one or more particular proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid molecules that are complementary to the complete sequences as reported in the accompanying Sequence Listing as well as those substantially similar nucleic acid sequences.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991), each of which, along with all other publications cited herein, is hereby incorporated by reference in its entirety. Methods to determine identity are codified in publicly available computer programs. Preferred computer program methods to determine identity between two sequences include, but are not limited to, the GCG Pileup program found in the GCG program package, as used in the instant invention, using the Needleman and Wunsch algorithm with their standard default values of gap creation penalty=12 and gap extension penalty=4 (Devereux, et al., 1984), BLASTP, BLASTN, and FASTA (Pearson, et al., 1988). The BLAST X program is publicly available from NCBI and other sources (BLAST Manual, Altschul et al., Natl. Cent. Biotechnol. Inf., Natl. Library Med. (NCBI NLM) NIH, Bethesda, Md. 20894; Altschul et al., 1990). Another preferred method to determine percent identity, is by the method of DNASTAR protein alignment protocol using the Jotun-Hein algorithm (Hein et al., 1990). Default parameters for the Jotun-Hein method for alignments are: for multiple alignments, gap penalty=11, gap length penalty=3; for pairwise alignments ktuple=6. As an illustration, by a polynucleotide having a nucleotide sequence having at least, for example, 95% “identity” to a reference nucleotide sequence of SEQ ID NO: 3 it is intended that the nucleotide sequence of the polynucleotide is identical to the reference sequence except that the polynucleotide sequence may include up to five point mutations per each 100 nucleotides of the reference nucleotide sequence of SEQ ID NO:3 In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid sequence of SEQ ID NO: 6 intended that the amino acid sequence of the polypeptide is identical to the reference sequence except that the polypeptide sequence may include up to five amino acid alterations per each 100 amino acids of the reference amino acid of SEQ ID NO:6. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

“Codon degeneracy” refers to the nature in the genetic code permitting variation of the nucleotide sequence without effecting the amino acid sequence of an encoded polypeptide. Accordingly, the instant invention relates to any nucleic acid molecule that encodes all or a substantial portion of the amino acid sequence encoding the instant SMT polypeptides as set forth in SEQ ID NOs:2 and 4. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene or gene copy that was not originally found in the host organism, but that is introduced into the host organism by gene transfer. Foreign genes can comprise native genes inserted into a non-native organism, additional copies of a native gene inserted into a native organism or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065;WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid molecule so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid molecule of the invention. Expression may also refer to translation of mRNA into a polypeptide.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals such as transit peptides.

A “signal peptide” is an amino acid sequence that is translated in conjunction with a protein and directs the protein across cell membranes of the cell in which the protein is made. For example, a signal peptide can be used to direct a mature SMT enzyme into a cell's vacuole via endoplasmic reticulum in accordance with the present invention. A signal peptide is also referred to as a signal protein. “Signal sequence” refers to a nucleotide sequence that encodes a signal peptide.

The present invention finds advantageous use in a wide variety of plants, as well as in a wide variety of microorganisms. With respect to plants, it is important to recognize that SMT has been found to become localized in vacuoles, and, therefore, that the polypeptide transcribed is a precursor protein which includes a signal peptide portion. The signal peptide is covalently bound to the “mature enzyme” or “passenger enzyme.” The term “precursor protein” identifies a polypeptide having a signal peptide and a passenger peptide covalently attached to each other. Typically, the carboxy terminus of the signal peptide is covalently attached to the amino terminus of the passenger peptide. The passenger peptide and signal peptide can be encoded by the same gene locus, that is, homologous to each other, in that they are encoded in a manner isolated from a single source. Alternatively, the signal peptide and passenger peptide can be heterologous to each other, i.e., the signal peptide and passenger peptide can be from different genes and/or different organisms. The signal peptide may be derived from monocotyledonous or dicotyledonous plants upon choice of the artisan. The term “signal peptide” includes amino acid sequences that are translated in conjunction with a protein and directs the protein to the secretory system (Chrispeels, 1991). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel, 1992).

By “mature peptide” or “passenger peptide” is meant a polypeptide which is found after processing and passing into an organelle and which is functional in the organelle for its intended purpose. Passenger peptides are originally made in a precursor form that includes a signal peptide and the passenger peptide. Upon entry into an organelle, the signal peptide portion is cleaved, thus leaving the “passenger” or “mature” peptide. Passenger peptides are the polypeptides typically obtained upon purification from a homogenate, the sequence of which can be determined as described herein.

“Transformation” refers to the transfer of a nucleic acid molecule into the genome of a host organism, resulting in genetically stable inheritance. Host organisms containing the transformed nucleic acid molecules are referred to as “transgenic” or “recombinant” or “transformed” organisms.

As used herein, “transgenic plant” includes reference to a plant which comprises within its genome a foreign polynucleotide. Generally, the foreign polynucleotide is stably integrated within the genome such that the polynucleotide is passed on to successive generations. The foreign polynucleotide may be integrated into the genome alone or as part of a recombinant expression cassette. “Transgenic” is used herein to include any cell, cell line, callus, tissue, plant part or plant, the genotype of which has been altered by the presence of foreign nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, WI), BLASTP, BLASTN, BLASTX (Altschul, et al., 1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

In one aspect, the invention provides nucleic acid fragments that encode functional SMT, and nucleic acid fragments substantially similar thereto. In alternative aspects, there are provided (1) a nucleotide sequence of the SMT gene, set forth in SEQ ID NO:5, (2) a nucleotide sequence encoding a precursor polypeptide including a signal peptide, having the nucleotide sequence set forth in SEQ ID NO:3, and (3) a nucleotide sequence encoding a mature SMT enzyme set forth in SEQ ID NO:5. Amino acid sequences encoded by these nucleic acid fragments are set forth in SEQ ID NO:4, SEQ ID NO:4 and SEQ ID NO:6, respectively.

A nucleic acid fragment having the sequence of SEQ ID NO:5 or of a substantially similar sequence can be operably coupled to a sequence encoding a signal peptide from a wide variety of species, including functionally similar variants thereof, to provide the advantageous result of the invention.

It is of course not intended that the present invention be limited to these exemplary nucleotide sequences, but the invention also encompasses nucleic acid fragments substantially similar to those set forth above. In a preferred aspect, the present invention provides nucleic acid fragments that encode functional polypeptides in accordance with the invention that have at least about 80% identity to the amino acid sequence of SEQ ID NO:2, SEQ ID NO:4 or SEQ ID NO:6, more preferably, at least about 90% identity to one of these sequences and most preferably at least about 95% identity. Another preferred aspect of the invention provides nucleic acid sequences corresponding to the instant SNG1 that are at least 80% identical to one of the nucleic acid sequences reported herein. More preferred nucleic acid fragments are at least 90% identical to one of the sequences herein. Most preferred are nucleic acid fragments that are at least 95% identical to one of the nucleic acid fragments reported herein.

For purposes of describing the invention, each of the above-described polypeptides is referred to hereafter as “SMT,” and each of the above-described nucleic acid fragments is referred to hereafter as “SNG1.”

In another aspect, the present invention relates to methods and compositions for obtaining transformed cells, said cells expressing SMT. In this regard, inventive nucleotide sequences can be incorporated into vectors, which in turn can be used to transform cells. Expression of SMT results in the cell having altered metabolic activity relative to nontransformed cells. Transformants harboring an expressible inventive nucleotide sequence demonstrate increased levels of sinapoylmalate production when appropriate substrates are available, and have other desirable features as would occur to a person of ordinary skill in the art. These and other features of the invention are described in further detail below.

Inventive DNA sequences can be incorporated into the genome of a plant or microorganism using conventional recombinant DNA technology, thereby making a transformed plant or microorganism that expresses SMT. As described above, the term “genome” as used herein is intended to refer to DNA which is present in a plant or microorganism and which is heritable by progeny during propagation thereof. As such, an inventive transformed plant or microorganism may alternatively be produced by producing F1 or higher generation progeny of a directly transformed plant or microorganism, wherein the progeny comprise the foreign nucleotide sequence. Transformed plants or microorganisms and progeny thereof are all contemplated by the invention and are all intended to fall directly within the meaning of the terms “transformed plant” and “transformed microorganism.”

In this manner, the present invention contemplates the use of transformed plants that are selfed to produce an inbred plant. The inbred plant produces seed containing the gene of interest. These seeds can be grown to produce plants that express the polypeptide of interest. The inbred lines can also be crossed with other inbred lines to produce hybrids. Parts obtained from the regenerated plant, such as flowers, seeds, leaves, branches, fruit, and the like are covered by the invention provided that said parts contain genes encoding and/or expressing the protein of interest. Progeny and variants, and mutants of the regenerated plants are also included within the scope of the invention.

In diploid plants, typically one parent may be transformed and the other parent is the wild type. After crossing the parents, the first generation hybrids (F1) are selfed to produce second generation hybrids (F2). Those plants exhibiting the highest levels of the expression can then be chosen for further breeding.

Standard recombinant DNA and molecular cloning techniques used in accordance with the present invention are well known in the art and are described by Sambrook, et al. (1989) (hereinafter “Maniatis”); and by Silhavy, et al. (1984); and by Ausubel, et al. (1987).

Recombinant Microbial Expression

It will be useful to recombinantly express the SNG1 gene for the recombinant production of SMT in heterologous host cells, particularly in the cells of microbial hosts, to produce large amounts of the SMT enzyme.

Preferred heterologous host cells for express of the instant genes and nucleic acid molecules are microbial hosts. Specific suitable hosts include but are not limited Aspergillus, Trichoderma, Saccharomyces, Pichia, Candida, Hansenula, Salmonella, Bacillus, Acinetobacter, Rhodococcus, Streptomyces, Escherichia, and Pseudomonas, where E. coli and Saccharomyces are most preferred.

Microbial expression systems and expression vectors containing regulatory sequences that direct high level expression of foreign proteins are well known to those skilled in the art. A wide variety of such systems and vectors could be used to construct chimeric genes for production of the gene products of the instant sequences. These chimeric genes could then be introduced into appropriate microorganisms via transformation to provide high level expression of the enzymes.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions are preferably derived from genes homologous to the transformed host cell, although it is to be understood that such control regions need not be derived from the genes native to the specific species chosen as a production host.

Initiation control regions or promoters, which are useful to drive expression of the instant genes in the desired host cell are numerous and familiar to those skilled in the art. Virtually any promoter capable of driving these genes is suitable for the present invention including but not limited to CYC1, HIS3, GAL1, GAL101, ADH, PGK, PHO5, GAPDH, ADC1, TRP1, URA3, LEU2, ENO, TPI (useful for expression in Saccharomyces); AOX1 (useful for expression in Pichia); and lac, ara, tet, trp, 1P_{L}, 1PR, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

It is readily understood that, in the case of transforming prokaryotes, it is not necessary to include a signal peptide in the coding region of the vector. Rather, an inventive DNA construct for transforming, for example, bacteria, may be made by simply attaching a start codon directly to, and in the proper reading frame with, a nucleic acid fragment encoding a mature peptide. For example, in one manner of practicing the invention, the vector includes a coding region having the sequence set forth in SEQ ID NO:5. Of course, other elements are preferably present as described herein, such as a promoter upstream of the start codon and a termination sequence downstream of the coding region.

Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal that is functional in the host production host. Methods for choosing appropriate signal sequences are well known in the art (see for example EP 546049;WO 9324631). The secretion signal DNA or facilitator may be located between the expression-controlling DNA and the instant gene or gene fragment, and in the same reading frame with the latter.

Expression in Transgenic Plants

The SNG1 gene may be used to create transgenic plants having the ability to express SMT. Transgenic plants expressing a functioning SNG1 gene are anticipated to exhibit modifications in their secondary metabolite profile. Similarly, SNG1 may be used in antisense orientation to alter or decrease secondary metabolite production in the plant.

Preferred plant hosts will be any variety that will support a high production level of the instant SMT proteins. Suitable green plants include but are not limited to soybean, rapeseed (Brassica napus, B. campestris), sunflower (Helianthus annus), cotton (Gossypium hirsutum), corn, tobacco (Nicotiana tabacum), alfalfa (Medicago sativa), wheat (Triticum sp), barley (Hordeum vulgare), oats (Avena sativa, L), sorghum (Sorghum bicolor), rice (Oryza sativa), Arabidopsis, cruciferous vegetables (broccoli, cauliflower, cabbage, etc.), melons, carrots, celery, parsley, parsnips, tomatoes, potatoes, strawberries, peanuts, grapes, grass seed crops, sugar beets, sugar cane, beans, peas, rye, flax, hardwood trees, softwood trees, and forage grasses.

The present invention further provides recombinant expression cassettes comprising the SNG1 coding region. A recombinant expression cassette will typically comprise a polynucleotide of the present invention (SNG1) operably linked to transcriptional initiation regulatory sequences which will direct the transcription of the SNG1 gene in the intended host cell, such as tissues of a transformed plant.

An expression vector according to the invention may be either naturally or artificially produced from parts derived from heterologous sources, which parts may be naturally occurring or chemically synthesized, and wherein the parts have been joined by ligation or other means known in the art. The introduced coding sequence is preferably under control of a promoter and thus will be generally downstream from the promoter. Stated alternatively, the promoter sequence will be generally upstream (i.e., at the 5′ end) of the coding sequence. The phrase “under control of” contemplates the presence of such other elements as may be necessary to achieve transcription of the introduced sequence. As such, in one representative example, enhanced production of SMT may be achieved by inserting an inventive nucleotide sequence in a vector downstream from and operably linked to a promoter sequence capable of driving expression in a host cell. Two DNA sequences (such as a promoter region sequence and an SMT-encoding nucleotide sequence) are said to be operably linked if the nature of the linkage between the two DNA sequences does not (1) result in the introduction of a frame-shift mutation, (2) interfere with the ability of the promoter region sequence to direct the transcription of the desired nucleotide sequence, or (3) interfere with the ability of the desired nucleotide sequence to be transcribed by the promoter region sequence.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal. A plant promoter fragment can be employed which will direct expression of SNG1 in all tissues of a generated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, and the GRP 1-8 promoter.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. Exemplary promoters include the anther specific promoter 5126 (U.S. Pat. Nos. 5,689,049 and 5,689,051), glob-1 promoter, and gamma-zein promoter. The operation of a promoter may also vary depending on its location in the genome. Thus, an inducible promoter may become fully or partially constitutive in certain locations.

Both heterologous and non-heterologous (i.e., endogenous) promoters can be employed to direct expression of SNG1 gene. These promoters can also be used, for example, in recombinant expression cassettes to drive expression of antisense nucleic acids to reduce, increase, or alter concentration and/or composition of the SMT protein in a desired tissue. Thus, in some embodiments, the nucleic acid construct will comprise a promoter functional in a plant cell, such as in Zea mays or tobacco, operably linked to SNG1. Promoters useful in these embodiments include the endogenous promoters driving expression of SMT.

In some embodiments, isolated nucleic acids that serve as promoter or enhancer elements can be introduced in the appropriate position (generally upstream) of a non-heterologous form of the SMT polynucleotide so as to up or down regulate its expression. For example, endogenous promoters can be altered in vivo by mutation, deletion, and/or substitution (see, Kmiec, U.S. Pat. No. 5,565,350; Zarling et al., PCT1 US93/03868), or isolated promoters can be introduced into a plant cell in the proper orientation and distance from SNG1 so as to control the expression of the gene. SNG1 expression can be modulated under conditions suitable for plant growth so as to alter the total concentration and/or alter the composition of SMT in a plant cell. Thus, the present invention provides compositions, and methods for making, heterologous promoters and/or enhancers operably linked to a native, endogenous (i.e., non-heterologous) form of SMT.

Where SMT polypeptide expression is desired, it is generally desirable to include a polyadenylation region at the 3′-end of a polynucleotide coding region of SNG1. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. (Buchman and Berg, 1988; Callis et al., 1987). Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot (1994). The vector comprising the SNG1 sequence will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers, et al. (1987).

SNG1 can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, SNG1 or a portion of SNG1 is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al. (1988); and Hiatt et al., U.S. Pat. No. 4,801,340.

Another method of suppression is sense suppression (i.e., co-supression). Introduction of nucleic acid configured in the sense orientation has been shown to be an effective means by which to block the transcription of target genes. For an example of the use of this method to modulate expression of endogenous genes see Napoli, et al. (1990), and U.S. Pat. No. 5,034,323. Such a method may be applied to the regulation of SNG1 expression.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff, et al. (1988).

To introduce SNG1 into a plant, generally the gene will first be incorporated into a recombinant expression cassette or vector, by a variety of methods known in the art. See, for example, Weising, et al, (1988). For example, the DNA construct may be introduced directly into the genomic DNA of the plant cell using techniques such as electroporation, polyethylene glycol (PEG), poration, particle bombardment, silicon fiber delivery, or microinjection of plant cell protoplasts or embryogenic callus. See, e.g., Tomes, et al, (1995). The introduction of DNA constructs using PEG precipitation is described in Paszkowski, et al. (1984). Electroporation techniques are described in Fromm, et al., 1985. Ballistic transformation techniques are described in Klein, et al. (1987).

Alternatively, Agrobacterium tumefaciens-mediated transformation techniques may be used. See, for example Horsch, et al., 1984; Fraley, et al., 1983; and, Plant Molecular Biology: A Laboratory Manual, Chapter 8, Clark, Ed., Springer-Verlag, Berlin (1997). The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller In: Genetic Engineering, vol. 6, PWJ Rigby, Ed., London, Academic Press, 1987; and Lichtenstein, C. P., and Draper, J, In: DNA Cloning, Vol. II, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman et al., 1984), (3) the vortexing method (see, e.g., Kindle, 1990).

DNA can also be introduced into plants by direct DNA transfer into pollen as described by Zhou, et al. (1983); Hess (1987); Luo, et al. (1988). Expression of SNG1 can be obtained by injection of the DNA into reproductive organs of a plant as described by Pena, et al. (1987). The gene can also be injected directly into the cells of immature embryos and the rehydration of desiccated embryos as described by Neuhaus, et al., (1987); and Benbrook, et al. (1986). A variety of plant viruses that can be employed as vectors are known in the art and include cauliflower mosaic virus (CaMV), geminivirus, brome mosaic virus, and tobacco mosaic virus.

Plant cells that directly result or are derived from the nucleic acid introduction techniques can be cultured to regenerate a whole plant that possesses the introduced genotype. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from a wide variety of plants can be successfully cultured to regenerate an entire plant. Plant regeneration from cultured protoplasts is described in Evans, et al. (1983); and Binding (1985).

The regeneration of plants from either single plant protoplasts or various explants is well known in the art. See, for example, Weissbach and Weissbach (1988). This regeneration and growth process includes the steps of selection of transformant cells and shoots, rooting the transformant shoots and growth of the plantlets in soil. For maize cell culture and regeneration see generally, Freeling and Walbot (1994); Sprague and Dudley (1988). For transformation and regeneration of maize see, Gordon-Kamm et al. (1990).

The regeneration of plants containing the polynucleotide of the present invention and introduced by Agrobacterium from leaf explants can be achieved as described by Horsch, et al (1985). In this procedure, transformants are grown in the presence of a selection agent and in a medium that induces the regeneration of shoots in the plant species being transformed as described by Fraley et al. (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Additional literature describing plant and/or microorganism transformation includes the following, each of which is incorporated herein by reference in its entirety: (Zhijian Li et al., 1992; Parsons, et al., 1997; Daboussi, et al., 1989; Leung, et al., 1990; Köbetter, et al., 1990; Strasser, et al., “Cloning of yeast xylose reductase and xylitol dehydrogenase genes and their use,” German patent application (1990); Hallborn, et al., 1991; Becker and Guarente, 1991; Ammerer, 1983; Sarthy, et al., 1987; U.S. Pat. Nos. 4,945,050, 5,141,131, 5,177,010, 5,104,310, 5,149,645, 5,469,976, 5,464,763, 4,940,838, 4,693,976, 5,591,616, 5,231,019, 5,463,174, 4,762,785, 5,004,863, 5,159,135, 5,302,523, 5,464,765, 5,472,869, 5,384,253; European Patent Application Nos. 0131624B1, 120516, 159418B1, 176112, 116718, 290799, 320500, 604662, 627752, 0267159, 0292435; WO 87/06614; WO 92/09696; and WO 93/21335.

Once the recombinant DNA is introduced into the plant tissue, successful transformants can be screened using standard techniques such as the use of marker genes, e.g., genes encoding resistance to antibiotics. Additionally, the level of expression of the foreign DNA may be measured at the transcriptional level, by measuring the amount of protein synthesized or by assaying to determine the level of enzyme function in the plant.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a polynucleotide of the present invention can be screened for transmission of the nucleic acid of the present invention by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are screened for the incorporated nucleic acid to identify and select plants with the most appropriate expression profiles.

Isolation/Identification of SNG1Homologs

The SNG1 gene and its SMT polypeptide gene product show a high percent identity to serine carboxypeptidases and serine carboxypeptidase-like (SCPL) enzymes. A comparison of the present nucleotide and amino acid sequences to the public databases indicated that there was a high degree of identity (30%-98%) to SCPL enzymes from Arabidopsis, Solanum, and Hordeum as shown in Table I below.

TABLE I
Comparison Of SMT Amino Acid Sequence To Known Serine
Carboxypeptidase-Like (SCPL) Enzymes

| | % | % | E- | |
|---|---|---|---|---|
| Similarity Identified | Identity^{a} | Positives | value^{b} | Citation |
| putative serine carboxypeptidase | 427/433 (98%) | 427/433 (98%) | 0.0 | Lin et al., Nature |
| I [Arabidopsis thaliana]. | | | | 402 (6763), 761- |
| ACCESSION AAC17816 | | | | 768 (1999) |
| glucose acyltransferase, putative | 291/417 (69%) | 344/417 (81%) | e-174 | Lin et al, |
| [Arabidopsis thaliana]. | | | | Unpublished |
| ACCESSION AAF76347 | | | | |
| glucose acyltransferase | 196/443 (44%) | 268/443 (60%) | 4e-98 | Li, A. X. and |
| [Solanum berthaultii]. | | | | Steffens, J. C., |
| ACCESSION AAD01263 | | | | Proc. Natl. Acad. |
| | | | | Sci. U.S.A. 97 |
| | | | | (12), 6902-6907 |
| | | | | (2000) |
| glucose acyltransferase | 190/439 (43%) | 276/439 (62%) | 8e-98 | Li, A. X. and |
| [Solanum berthaultii]. | | | | Steffens, J. C., |
| ACCESSION AAD01264 | | | | Proc. Natl. Acad. |
| | | | | Sci. U.S.A. 97 |
| | | | | (12), 6902-6907 |
| | | | | (2000) |
| glucose acyltransferase | 195/443 (44%) | 267/443 (60%) | 2e-96 | Li, A. X. and |
| [Solanum berthaultii]. | | | | Steffens, J. C., |
| ACCESSION AAD01265 | | | | Proc. Natl. Acad. |
| | | | | Sci. U.S.A. 97 |
| | | | | (12), 6902-6907 |
| | | | | (2000) |
| serine carboxypeptidase i | 104/253 (41%) | 154/253 (60%) | 4e-53 | Doan et al, J. Biol. |
| precursor (carboxypeptidase c) | | | | Chem. 263 (23), |
| (CP-MI). | | | | 11106-11110 |
| ACCESSION P07519 | | | | (1988) |
| putative serine carboxypeptidase | 136/442 (30%) | 207/442 (46%) | 2e-46 | Lin et al., Nature |
| II [Arabidopsis thaliana]. | | | | 402 (6763), 761- |
| ACCESSION AAD21479 | | | | 768 (1999) |

^{a}% Identity is defined as percentage of amino acids that are identical between the two proteins..
^{b}Expect value. The Expect value estimates the statistical significance of the match, specifying the number of matches, with a given score, that are expected in a search of a database of this size absolutely by chance.

In another aspect of the invention, the sequence of the SNG1 gene may be used to isolate genes encoding homologous proteins from other plants, which genes, and the expression products thereof, can be readily tested for functionality in accordance with the present invention by a person of ordinary skill in the art. It is well known that plants and microorganisms of a wide variety of species commonly express and utilize analogous enzymes and/or polypeptides which have varying degrees of degeneracy, and yet which effectively provide the same or a similar function. For example, an amino acid sequence isolated from one species may differ to a certain degree from the wild-type sequence set forth in SEQ ID NO:4, and yet have similar functionality. Amino acid sequences comprising such variations, and methods for identifying and isolating the same, are included within the scope of the present invention.

Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g polymerase chain reaction (PCR), Mullis, et al., U.S. Pat. No. 4,683,202; ligase chain reaction (LCR), Tabor, et al. (1985); or strand displacement amplification (SDA), Walker, et al. (1992)).

For example, genes encoding similar proteins or polypeptides to the SMT enzyme could be isolated directly by using all or a portion of the instant nucleic acid molecules as DNA hybridization probes to screen libraries from any desired bacteria using methodology well known to those skilled in the art. Specific oligonucleotide probes based upon the instant SNG1 sequences can be designed and synthesized by methods known in the art (Maniatis). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length DNA fragments under conditions of appropriate stringency.

Generally two short segments of the instant sequences may be used in polymerase chain reaction protocols to amplify longer nucleic acid molecules encoding homologous genes from DNA or RNA. The polymerase chain reaction may also be performed on a library of cloned nucleic acid molecules wherein the sequence of one primer is derived from the instant SNG1 nucleic acid molecules, and the sequence of the other primer takes advantage of the presence of the polyadenylic acid tracts to the 3′ end of the mRNA precursor encoding the instant genes. Alternatively, the second primer sequence may be based upon sequences derived from the cloning vector. For example, the skilled artisan can follow the RACE protocol (Frohman, et al., 1988) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara, et al., 1989; Loh, et al., 1989). Typically, in PCR-type amplification techniques, the primers have different sequences and are not complementary to each other. Depending on the desired conditions, the sequences of the primers should be designed to provide for both efficient and faithful replication of the target nucleic acid. Methods of PCR primer design are common and well known in the art (Thein and Wallace, 1986; Rychlik, 1993).

Alternatively the instant SNG1 sequences may be employed as hybridization reagents for the identification of homologs. The basic components of a nucleic acid hybridization test include a probe, a sample suspected of containing the gene or gene fragment of interest, and a specific hybridization method. Probes of the present invention are typically single stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, 1991). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1M sodium chloride, about 0.05 to 0.1M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents that include a variety of polar water-soluble or swellable agents, such as polyethylene glycol, anionic polymers such as polyacrylate or polymethylacrylate, and anionic saccharidic polymers, such as dextran sulfate.

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

Availability of the instant nucleotide and deduced amino acid sequences facilitates immunological screening of DNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen DNA expression libraries to isolate full-length DNA clones of interest (Lemer, 1984; Maniatis).

It is also contemplated in accordance with the present invention that SNG1 can be used to produce gene products having enhanced or altered activity. Various methods are known for mutating a native gene sequence to produce a gene product with altered or enhanced activity including but not limited to error prone PCR (Melnikov, et al., 1999); site directed mutagenesis (Coombs et al., 1998), and “gene shuffling” (U.S. Pat. No. 5,605,793; No. 5,811,238; No. 5,830,721; and No. 5,837,458, incorporated herein by reference).

The method of gene shuffling is particularly attractive due to its facile implementation, and high rate of mutagenesis and ease of screening. The process of gene shuffling involves the restriction endonuclease cleavage of a gene of interest into fragments of specific size in the presence of additional populations of DNA regions of both similarity to and difference from the gene of interest. This pool of fragments will then be denatured and reannealed to create a mutated gene. The mutated gene is then screened for altered activity.

The instant SNG1 sequences can be mutated and screened for altered or enhanced activity by this method. The sequences should be double stranded and can be of various lengths ranging form 50 bp to 10 kb. The sequences can be randomly digested into fragments ranging from about 10 bp to 1000 bp, using restriction endonucleases well known in the art (Maniatis supra). In addition to the instant SNG1 sequences, populations of fragments that are hybridizable to all or portions of the SNG1 sequence can be added. Similarly, a population of fragments that are not hybridizable to the instant SNG1 sequence can also be added. Typically these additional fragment populations are added in about a 10 to 20 fold excess by weight as compared to the total nucleic acid. Generally if this process is followed the number of different specific nucleic acid fragments in the mixture will be about 100 to about 1000. The mixed population of random nucleic acid fragments are denatured to form single-stranded nucleic acid fragments and then reannealed. Only those single-stranded nucleic acid fragments having regions of homology with other single-stranded nucleic acid fragments will reanneal. The random nucleic acid fragments may be denatured by heating. One skilled in the art could determine the conditions necessary to completely denature the double stranded nucleic acid. Preferably the temperature is from 80° C. to 100° C. The nucleic acid fragments may be reannealed by cooling. Preferably the temperature is from 20° C. to 75° C. Renaturation can be accelerated by the addition of polyethylene glycol (“PEG”) or salt. A suitable salt concentration may range from 0 mM to 200 mM. The annealed nucleic acid fragments are next incubated in the presence of a nucleic acid polymerase and dNTP's (i.e. dATP, dCTP, dGTP and dTTP). The nucleic acid polymerase may be the Klenow fragment, the Taq polymerase or any other DNA polymerase known in the art. The polymerase may be added to the random nucleic acid fragments prior to annealing, simultaneously with annealing or after annealing. The cycle of denaturation, renaturation and incubation in the presence of polymerase is repeated for a desired number of times. Preferably the cycle is repeated from 2 to 50 times, more preferably the sequence is repeated from 10 to 40 times. The resulting nucleic acid is a larger double-stranded polynucleotide of from about 50 bp to about 100 kb and may be screened for expression and altered activity by standard cloning and expression protocol. (Maniatis supra).

Methods of Use: Altering Plant Metabolism

The SNG1 gene has broad applicability for the modification of plant metabolism, and traits related to plant metabolism. Enzymes known to make glucose conjugates are well known in plants and have been shown to act on a wide variety of substrates (Corner, et al., 1965; Lim, et al., 2001). Similarly, SMT has been shown to accept a broad range of substrates (Strack and Sharma, 1985). Suitable substrates known to a skilled artisan include benzoic acid, o-hydroxybenzoic acid, m-hydroxybenzoic acid, 3,4-dihydroxybenzoic acid, vanillic acid, syringic acid, cinnamic acid, o-coumaric acid, m-coumaric acid, caffeic acid, ferulic acid, 5-hydroxyferulic acid, isoferulic acid, and sinapic acid. Thus, incorporating the SMT gene into a transformed plant by means known in the art will result in the expression of the SMT protein, and the activity of the SMT protein will lead to the conversion of monosaccharide esters, such as glucose esters, into their corresponding malate esters. SCPL proteins function in a broad range of biochemical pathways, including those of secondary metabolite biosynthesis, herbicide conjugation, and germination-associated degradataion of seed protein reserves. Thus, these proteins are vital for normal plant growth and development, for the synthesis of compounds that protect plants against pathogens and UV light, and for resistance to natural and manmade xenobiotics. The identification of SMT as a SCPL protein has cast new light on the potential of these enzymes to serve as participants in diverse biochemical pathways. For example, certain glucose esters are metabolically related to lignin biosynthetic intermediates (Whetten, et al., 1998), the redirection of these metabolites into their corresponding malic acid esters by the reaction catalyzed by SMT will remove them from the lignin biosynthetic pathway. Examples of such glucose esters include monosaccharide esters of cinnamic acid, p-coumaric acid, caffeic acid, ferulic acid, 5-hydroxyferulic acid and sinapic acid. Thus, it will be possible to modify lignin content and composition by the overexpression of the SMT gene in lignifying tissues. As a further example of a useful SMT, transacylation of monosaccharide esters, such as glucose esters, is known to be useful for providing insect resistance in plants (Ghanges and Steffens, 1995). In addition, since the reaction catalyzed by SMT is primarily dependent upon the free energy provided by the 1-O-acylglucosidic bond (Mock and Strack, 1993), it will be obvious to a person skilled in the art that alternative sugar esters would be able to provide similar energy for the SMT catalyzed reaction. Thus, esters of other monosaccharides including without limitation, ribulose, sylulose, psicose, fructose, sorbose, tagatose, sedoheptulose, ribose, arabinose, xylose, lyxose, allose, altrose, mannose, gulose, idose, galactose, and talose would be suitable substrates for SMT of the present invention. Other possible substrates and sugar esters which can be used in the practice of the present invention will be known to those skilled in the art.

Those skilled in the art would recognize that the absence of obvious phenotypes in mutants lacking sinapoylcholine is also a finding of potential agronomic importance (Chapple, et al., 1992). Oilseed rape or canola (Brassica sp.) accumulates sinapoylcholine in seeds, and when post-crushing canola meal is used in poultry feed, by-products of sinapoylcholine degradation impart a fishy taint to eggs (Hobson-Frohock, et al., 1977). The examination of breeding lines of B. napus and B. campestris for genetic variation in seed sinapoylcholine accumulation has not identified significant variation for the trait (Vogt, et al., 1993). The isolation of the sinapoylcholine-deficient fah1 mutant strongly suggests that it should be possible to manipulate sinapoylcholine levels in Brassica crops, and the cloning of the SNG1 gene provides the tools necessary for a genetic engineering approach to this problem. It will be obvious to a person trained in the art that expression of SMT in a transformed plant seed will compete with the biosynthesis of sinapoylcholine because both sinapoylcholine and sinapoylmalate are synthesized from a common precursor, sinapoylglucose. Thus, it will be possible to decrease sinapoylcholine biosynthesis by ectopic overexpression of the SMT gene.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

Standard recombinant DNA and molecular cloning techniques used in the Examples are well known in the art and are described by Sambrook, et al. (1989); Maniatis; Silhavy (1984) and by Ausubel, et al (1987).

Materials and methods suitable for the maintenance and growth of bacterial cultures are well known in the art. Techniques suitable for use in the following examples may be found as set out in Manual of Methods for General Bacteriology (Gerhardt, et al., 1994) or by Thomas D. Brock in Biotechnology: A Textbook of Industrial Microbiology (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis). Where the GCG program “Pileup” was used the gap creation default value of 12, and the gap extension default value of 4 were used. Where the CGC “Gap” or “Bestfit” programs were used the default gap creation penalty of 50 and the default gap extension penalty of 3 were used. In any case where GCG program parameters were not prompted for, in these or any other GCG program, default values were used.

Plant Material

Arabidopsis thaliana L. Heynh. ecotypes Columbia or Landsberg erecta were cultivated at a light intensity of 100 μE m⁻²sec⁻¹ at 23° C. under a photoperiod of 16 h light/8 h dark in ProMix potting mixture. For seedling plant material to be used in the analysis of SNG1 mRNA accumulation, seeds were surface-sterilized for 30 minutes in a 2:1 mixture of 0.1% Triton X-100 and household bleach. Seeds were rinsed thoroughly with sterile water and plated on Miracloth (Calbiochem) discs on modified-MS medium (ammonia-free medium to which an additional 20.6 mM potassium nitrate was added in place of ammonium nitrate) (Murashige and Skoog, 1962) containing 0.7% agar.

Secondary Metabolite Analysis

Leaf extracts were prepared from 100 mg samples of fresh leaf tissue suspended in 1 mL of 50% methanol. Samples were ground briefly, then centrifuged at 12,000× g for 5 minutes. Sinapate ester content was qualitatively determined by UV fluorescence following chromatography of extracts on silica gel TLC plates in a mobile phase of n-butanol:ethanol:water 4:1:1, or quantitatively determined by HPLC.

HPLC Analysis

Plant extracts and SMT assays were analyzed by HPLC on a Nova-Pak® C18 column (Waters) (60 Å pore size, 4μ particle size) using a 15 minute gradient at 1 mL min⁻¹ from 6% acetonitrile, 1.5% phosphoric acid to 48% acetonitrile, 1.5% phosphoric acid and UV detection at 335 nm.

Analysis of Nucleic Acids

For DNA gel blot analyses, DNA was extracted from leaf material (Rogers and Bendich, 1985), digested with restriction endonucleases, electrophoretically separated, transferred to Hybond N⁺ membrane (Amersham) and hybridized with cDNA probes according to standard protocols (Sambrook et al., 1989). RNA was extracted from tissues (Goldsbrough and Cullis, 1981), electrophoretically separated, transferred to Hybond N membrane (Amersham), and hybridized with radiolabelled probes prepared from genomic clones according to standard protocols. Sequencing of genomic and cDNA clones was performed on a Pharmacia ALFexpress automated DNA sequencer (Uppsala, Sweden) using standard primers.

The meaning of abbreviations is as follows: “h” means hour(s), “min” means minute(s), “sec” means second(s), “d” means day(s), “mL” means milliliters, “L” means liters.

### Example 1

### Isolation Cloning, Sequencing and Characterization of SNG1

The fluorescence of sinapoylmalate accumulated in the epidermis of Arabidopsis leaves can be visualized in vivo when wild-type plants are observed under UV light (Chapple, et al., 1992; Ruegger, et al., 1999). To observe and record the appearance of wild-type and sng1 mutant plants under UV light, they were observed and photographed using a 365-nm transilluminator as a light source, Ektachrome 160 film, and a Toshiba Y2 yellow glass barrier filter to remove reflected UV light. Mature sng1 mutants contain levels of sinapoylglucose that are comparable to the sinapoylmalate concentration found in wild-type plants; however, they are less fluorescent than their wild-type counterparts. The diminished fluorescence phenotype of the sng1 mutant was used to identify two independent sng1 alleles (sng1-3 and sng1-4) among the T-DNA tagged Arabidopsis lines available from the Arabidopsis Biological Resource Center at The Ohio State University. Back-crosses to the wild-type ecotype Wassilewskija followed by tests of co-segregation demonstrated that the kanamycin-resistant phenotype engendered by the T-DNA cosegregated with the mutant phenotype of sng1-4, but not sng1-3.

To provide additional resources for cloning the SNG1 gene, a number of sng1 alleles (sng1-5 through sng1-8) were identified from fast neutron-mutagenized populations of Arabidopsis. Fast neutrons are known to generate deletions; thus, lines that have a sng1 phenotype would be likely to carry restriction fragment length polymorphisms that would be helpful in the map-based cloning of SNG1. From a screen of 42,000 plants representing 12 parental groups, four independent mutants were identified with UV and TLC phenotypes similar to sng1. The biochemical phenotype of these mutants was verified by HPLC analysis, and all of the mutants failed to complement sng1-1, indicating that these plants carry new sng1 alleles.

Inverse PCR was employed to amplify the genomic DNA adjacent to the T-DNA insertion in sng1-4 using primers designed against the known sequence of the T-DNA vector. To isolate regions flanking the T-DNA insert in the sng1-4 mutant, genomic DNA was extracted as described above. Genomic DNA was digested with BclI and circularized with T4 DNA ligase. Inverse PCR was carried out using the primers 5′-GATGCACTCGAAATCA-GCCA-3′ (SEQ ID NO:7) and 5′-GCGCGGAGTCATTACAGTTA-3′ (SEQ ID NO:8) employing 35 one minute cycles and a primer annealing temperature of 54° C. From these reactions, a single 768 bp fragment was amplified. The fragment was used to screen a cosmid library constructed in the transformation-competent binary vector pBIC20 (Meyer, et al., 1996). SNG1 cDNA and genomic clones were identified by standard techniques (Sambrook, et al., 1989) using the inverse PCR fragment amplified from sng1-4 as a probe. The SNG1 cDNA clone was identified in a library prepared from ten day old abi1 seedling mRNA (Meyer, et al., 1994). The SNG1 genomic clones were identified in an Arabidopsis thaliana (ecotype Landsberg erecta) library generated in the binary cosmid vector pBIC20 (Meyer, et al., 1996). Three classes of cosmids were recovered by this screening as determined by digestion with HindIII. All three classes shared a common 3.9 kb fragment that hybridized with the inverse PCR product in DNA gel blot analysis.

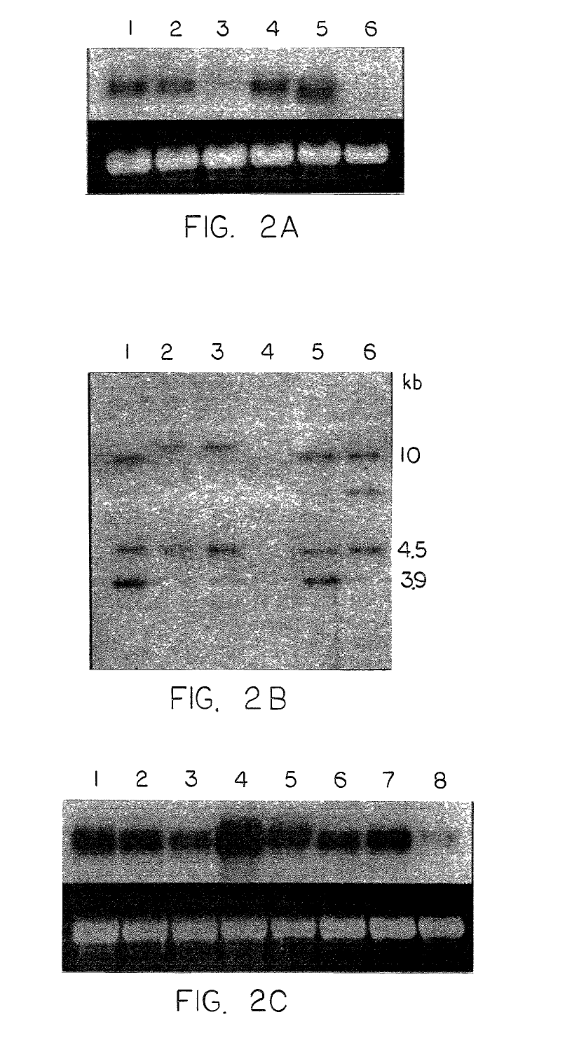

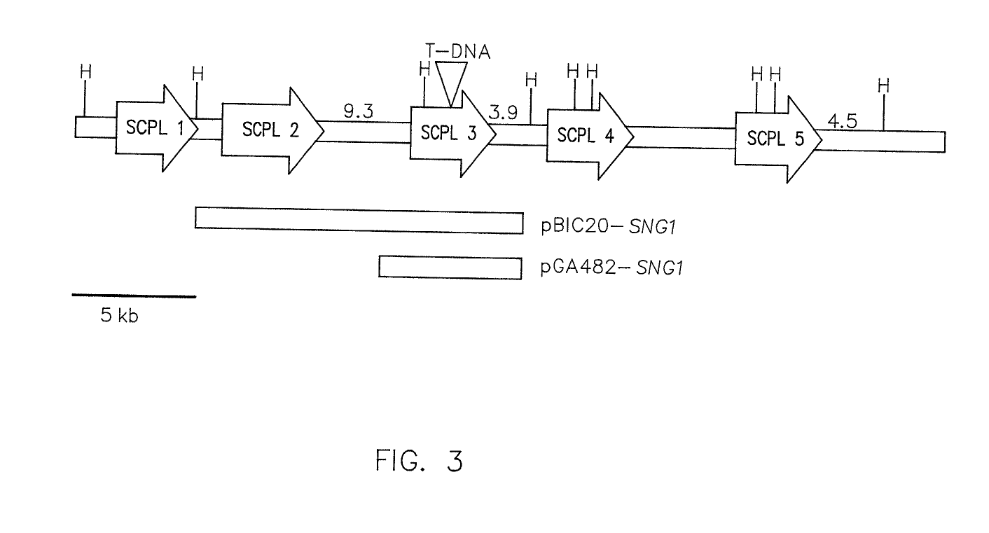

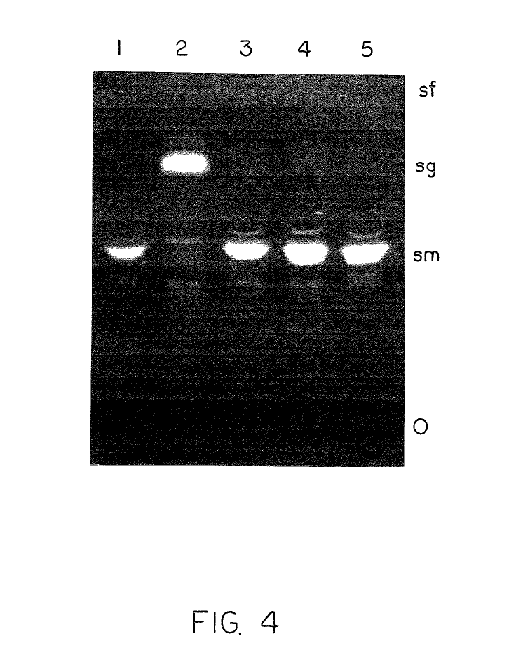

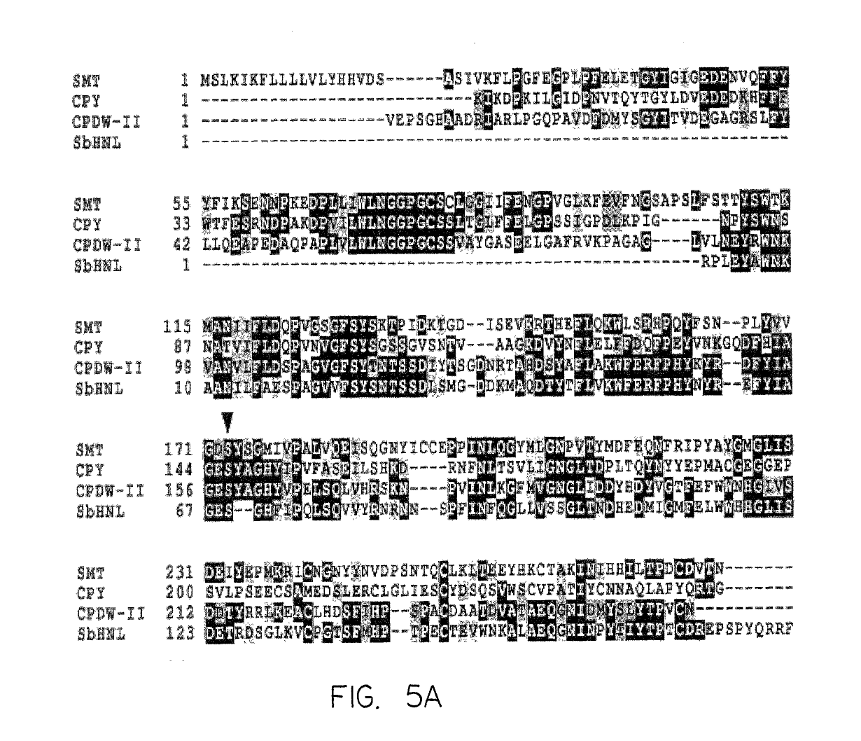

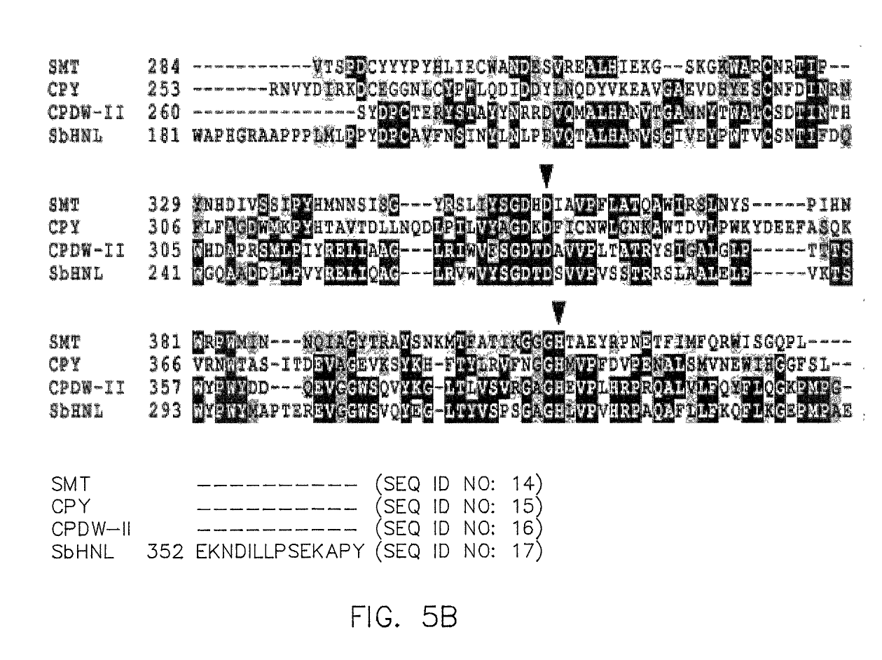

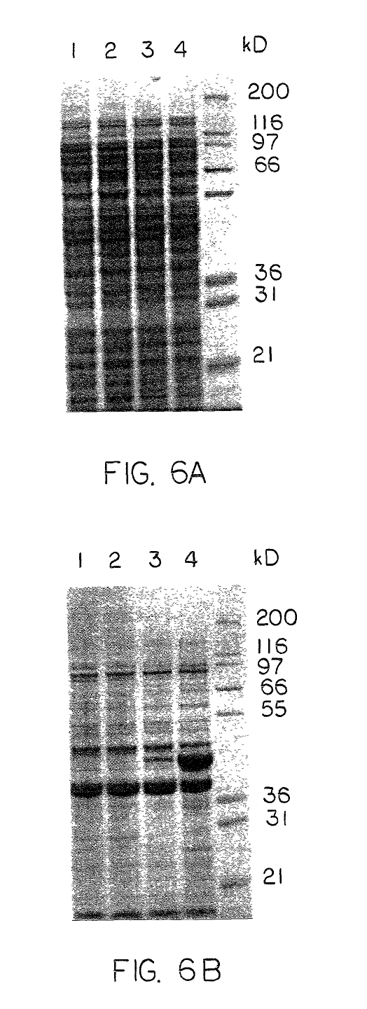

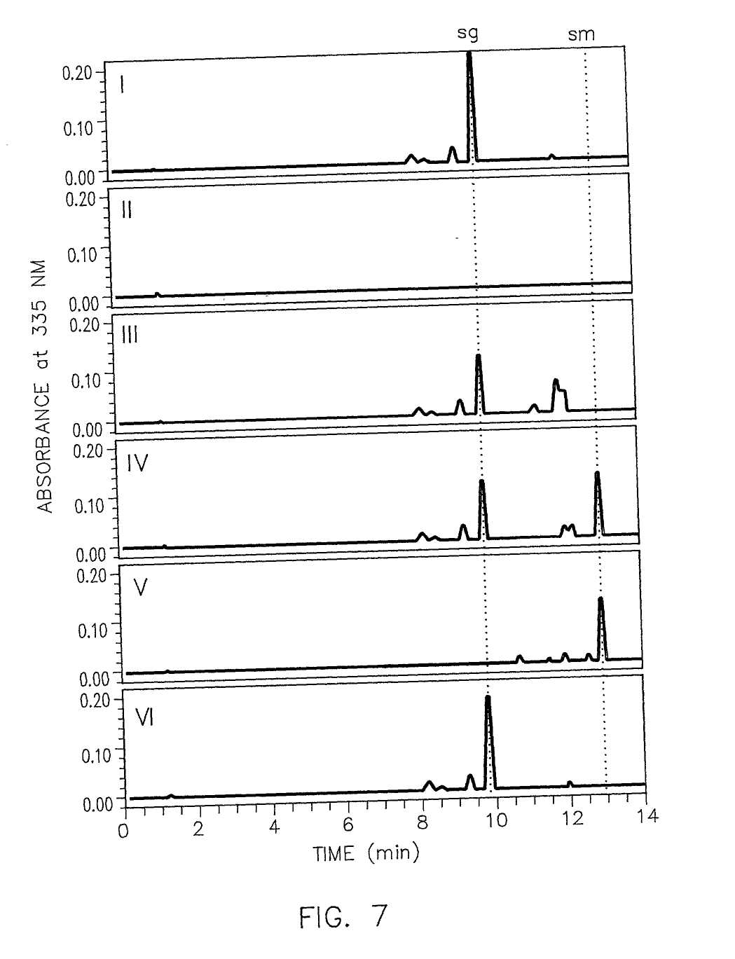

Before attempting to complement the sng1 mutant, two independent approaches were used to determine whether the 3.9 kb fragment shared by these cosmids was likely to carry at least a portion of the SNG1 gene. First, the 3.9 kb fragment was used to identify potential SNG1 transcripts and to compare their abundance in plants homozygous for each of four sng1 alleles (FIG. 2). RNA gel blot hybridization analysis identified a potential SNG1 transcript that was present at similar levels in leaf tissue of Columbia and Wassilewskija ecotypes. Transcript was present at wild-type levels in the lines homozygous for sng1-2 and sng1-3, although the transcript in sng1-3 may be truncated. Transcript abundance was substantially reduced in the EMS-induced sng1-1 mutant and was below detectable limits in the T-DNA tagged line, sng1-4. Considering that EMS-induced mis-sense mutations and insertional mutagenesis often lead to mRNA destabilization, these data provided correlative evidence that we had cloned the SNG1 gene.

Next, the fast neutron-induced sng1 alleles were used to determine whether these lines exhibited DNA polymorphisms associated with the putative SNG1 locus. These experiments demonstrated that three of the four mutant lines carried deletions large enough to be detected by DNA gel blot analysis, and of those three, all had deletions that affected or eliminated hybridization of the 3.9 kb HindIII fragment to their genomic DNA. These data provide additional support that the SNG1 protein is at least partially encoded by this DNA. Based upon the sequence data described below, one cosmid (FIG. 4; hereafter referred to as pBIC20-SNG1) was characterized further.

The 3.9 kb HindIII restriction fragment of pBIC20-SNG1 (FIG. 3) was subcloned and sequenced. Blastx analysis (Altschul, et al., 1990) indicated that this region of the genome was likely to encode a protein with homology to serine carboxypeptidase proteins (score 48, E value 5×e−5, closest homologue carboxypeptidase I precursor from Hordeum vulgare, Genbank accession number J03897). To examine the expression of this putative carboxypeptidase, an RNA gel blot was probed with the 3.9 kb fragment from pBIC20-SNG1 (FIG. 2). Although sinapoylmalate is accumulated primarily in leaves of Arabidopsis and related crucifers, expression of the putative SNG1 gene was observed in almost all tissues examined. The highest level of message observed in 10-day-old seedlings and only a low level of SNG1 mRNA was found in roots.

To further characterize pBIC20-SNG1, the 9.3 kb HindIII fragment upstream of the 3.9 kb fragment (FIG. 3) was subcloned and partially sequenced. As expected, Blastx analysis of the 3′ end of the fragment (as defined relative to the direction of the putative SNG1 open reading frame) returned homology to serine carboxypeptidase proteins. This was consistent with the previous analysis of the 5′ end of the downstream 3.9 kb fragment which showed homology to internal sequences of serine carboxypeptidase proteins. Surprisingly, analysis of the 5′ end of the 9.3 kb fragment also indicated that this region encodes a serine carboxypeptidase-like (SCPL) protein. These data provided the first suggestion that at least two SCPL proteins are encoded near the SNG1 locus.

### Example 2

### pBIC20-SNG1 Complements the sng1 Mutant Phenotype

To provide definitive proof that pBIC20-SNG1 carries the SNG1 genomic sequence, this cosmid was introduced into Agrobacterium tumefaciens C58 pGV3850 (Zambrisky, et al., 1983) by electroporation, and cultures harboring the binary vector were used to transform the sng1-1 mutant. Plant transformation was performed by vacuum infiltration (Bent, et al., 1994) with minor modifications (Bell-Lelong, et al., 1997). Transformed seedlings (T₁) were identified by selection on MS medium containing 50 mg L⁻¹ kanamycin and 200 mg L⁻¹ timentin. Thirty-four kanamycin-resistant seedlings representing nineteen independent transformation events were transferred to soil and tested for their profile of sinapate esters by TLC. All plants contained sinapoylmalate instead of, or as well as, sinapoylglucose, indicating total or partial complementation of the mutant phenotype (FIG. 4). These data unequivocally demonstrated that the gene that is defective in the sng1 mutant is encoded on the pBIC20-SNG1 cosmid.

TAMU BAC F21P24 was being sequenced by the Arabidopsis Genome Initiative at the time the initial sequence data for pBIC20-SNG1 was obtained. When the complete BAC sequence was released, it revealed that BAC F21P24 carries the SNG1 locus and five SCPL genes surrounding the SNG1 gene (FIG. 3). One of these genes (not shown in FIG. 3) has been annotated in the database as a pseudogene because the region corresponding to its first exon is flanked by sequences with high similarity to ATPases, suggesting that this SCPL gene lacks a promoter. All of the SCPL proteins encoded by this region of the genome are highly similar to one another. Comparison of their deduced amino acid sequences indicates that they share between 69 and 78% amino acid identity. Their similarity and tandem arrangement suggests that they may be the result of relatively recent gene duplication events. RNA gel blot hybridization experiments indicate that these genes are expressed only at very low levels in all tissues examined previously for SNG1 expression. These data also indicate that the widespread expression previously observed for SNG1 is not an artefactual result of cross-hybridization to mRNA of these other SCPL genes.

The partial sequence data, and the release of the sequence of BAC F21P24 demonstrated that pBIC20-SNG1 encodes two SCPL proteins (FIG. 3). To unambiguously prove which gene is defective in the sng1 mutant a new construct (pGA482-SNG1) was generated that contains only the downstream SCPL gene under the control of 1.1 kb of its upstream regulatory sequence and used it to transform the sng1 mutant. For generation of the pGA482-SNG1 construct, a region corresponding to the SNG1 promoter was amplified by PCR using the upstream primer 5′-CGGGTACCAGCAAAACGCATC-AACCATAAAC-3′ (SEQ ID NO:9) and the downstream primer 5′-GAGGGCCGGGACAATCATA-3′ (SEQ ID NO:10). The upstream primer introduces a new KpnI site into the sequence, and the downstream primer binds downstream of the HindIII site that is internal to the SNG1 gene. The amplification product was subcloned into pGEMT-Easy (Promega) for sequencing, and then liberated with KpnI and HindIII for subcloning into similarly digested pGA482 (An, 1987). The resulting vector was then digested with HindIII, and the 3.9 kb HindIII fragment from pBIC20-SNG1 was inserted and checked for orientation by PCR to generate pGA482-SNG1. Like pBIC20-SNG1, the genomic sequence carried on pGA482-SNG1 complements the sng1 phenotype indicating that we have identified the SNG1 gene (SCPL 3 in FIG. 3).

### Example 3

### Expression of SNG1 E. coli

The 3.9 kb fragment of pBIC20-SNG1 was used to screen a cDNA library prepared from ten day old Arabidopsis seedlings (Meyer, et al, 1994). A number of clones were retrieved, the longest of which was sequenced. The predicted N-terminal sequence was analyzed using the algorithm described by Nielsen et al. (Nielsen, et al., 1997, and available at the SignalP website (www.cbs.dtu.dk/services/SignalP/). Results indicated the presence of a signal peptide that is likely to be cleaved after residue S19. If this prediction were correct, the inferred translation product of 49.4 kD would give rise to a mature protein with a mass of 47.2 kD. Analysis of the SMT sequence using the PSORT algorithm (psort.nibb.ac jp/) predicted six possible glycosylation sites, and indicated that the protein is most likely localized in the vacuole. These predictions are consistent with previous research that demonstrated SMT to be a vacuolar protein (Strack and Sharma, 1985). Most importantly, the sequence ASIVKFLPGFEGPLPFE (SEQ ID NO:11) was found immediately following the predicted cleavage site. This peptide matched, at 16 of 17 residues, the N-terminal sequence that was obtained when SMT was purified from Brassica napus as described by Grawe, et al. (1992), blotted onto PVDF membrane, and sequenced using a model 120A liquid phase protein sequencer (data not shown).

Comparison of the inferred amino acid sequence of the cDNA to the database indicated substantial similarities with SCPL proteins from plants, animals, and yeast (FIG. 5). The inferred amino acid sequence of the putative SMT cDNA shares 18% identity with carboxypeptidase Y from Saccharomyces cerevisiae and 23% identity with the wheat carboxypeptidase for which the crystal structure has been determined (Liao and Remington, 1990; Liao, et al., 1992) (FIG. 6). The putative SMT sequence shares the conserved serine, aspartic acid, and histidine residues (S173, D358, and H411 in the SMT sequence) that have been demonstrated through inhibitor studies and site-directed mutagenesis (Hayashi, et al., 1973;Hayashi, et al., 1975; Bech and Breddam, 1989), as well as crystallographic analysis (Liao and Remington, 1990; Liao, et al., 1992), to comprise the catalytic triad that is essential for enzymatic activity. The involvement of an active site serine residue in the SMT protein is supported by the observation that pre-incubation with phenylmethylsulfonyl fluoride inhibited by 30% the activity of SMT extracted from Arabidopsis leaves.

Although these data provided strong evidence that the SNG1 gene encodes SMT, the possibility that SNG 1 is a serine carboxypeptidase required for the proteolytic activation of one or more vacuolar pro-proteins that would include SMT was not yet excluded. Indeed, this interpretation could be supported by our findings that SNG1 transcript is expressed in tissues other than those known to accumulate sinapoylmalate. In addition, the five SCPL genes clustered at the SNG1 locus on chromosome 2 encode proteins with high amino acid identity in their amino terminal region (only one to three amino acid substitutions) with the N-terminal sequence of SMT purified from B. napus. Consequently this amino acid sequence is not necessarily diagnostic for SMT. Finally, although carboxypeptidases have been shown to catalyze acyltransferase reactions under non-physiological conditions (Widmer and Johansen, 1979; Widmer, et al., 1980), there are no precedents in the literature for SCPL proteins acting as acyltransferases in vivo.

To unequivocally determine whether the SNG1 gene encodes SMT, the SNG1 cDNA was expressed in E. coli (FIG. 6). For these experiments, the portion of the SNG1 open reading frame corresponding to the mature N-terminally trimmed polypeptide of SEQ ID NO:6 was subcloned into pET28A under the control of the T7 promoter as follows.

Two oligonucleotides designed to amplify a fragment of the SNG1 cDNA encoding a protein lacking the predicted signal peptide were used to create a fragment suitable for cloning into the pET28A expression vector (Novagen). The N-terminal oligonucleotide 5′-TCATGACCTCTATCGTCAAGTTTCTTC C-3′ (SEQ ID NO: 12) incorporated a start codon and the restriction site PagI (TCATGA), and altered the N-terminal alanine codon (GCC) to a threonine codon (ACC). The C-terminal oligonucleotide 5′-GTCGACTTACAGGGGTTGGCCACTG-3′ (SEQ ID NO:13) incorporated a SalI restriction site after the stop codon. The SNG1 gene was amplified by PCR, subcloned and sequenced. The SNG1 gene was excised by PagI-Sall digestion and cloned into the NcoI-SalI digested pET28A vector to yield pET28A-SNG1. For analysis of SNG1 expression and activity, the E. coli host BL21DE3 was transformed with the empty pET28A vector and pET28A-SNG1.

For heterologous expression of SNG1, an overnight culture of bacteria grown at 37° C. was diluted 200-fold into fresh LB medium and grown at 18° C. to an OD₆₀₀ₙₘ of 0.6. Cells were subsequently induced with 0.8 mM IPTG and grown for 48 h at 14° C. Cells were harvested and lysed in 2.5 mL of 20 mM Tris/HCl (pH 8), 500 mM NaCl using a french press. The cell lysate was cleared by centrifugation at 14,000× g at 4° C. for 30 minutes. Supernatant (soluble protein fraction) and pellet (insoluble protein fraction) were analyzed by SDS-PAGE. Protein concentration of the soluble fraction was determined using the Bradford assay (Bradford MM (1976) A rapid and sensitive method for the quantitation of microgram quantities of protein utilizing the principle of protein-dye binding. Anal Biochem 72: 248-254).

When analyzed by SDS-PAGE, no obvious differences were observed between the soluble proteins extracted from cells carrying pET28A and pET28A-SNG1 grown at 14° C. in either the presence or absence of IPTG (FIG. 6). In contrast, a distinct novel band with a molecular mass of approximately 44 kD was visible in uninduced and induced cells carrying the pET28A-SNG1 vector. The size of this band was somewhat less than, but reasonably consistent with the expected size of the SNG1 protein. Although these data indicated that the bulk of the SNG 1 protein was present in inclusion bodies, we assayed samples of the supernatant protein for SMT activity as follows.

### Example 4

### Assay of SMT Activity

It was expected that if only a small percentage of the protein was correctly folded and soluble, its enzymatic activity could be readily measured even though the protein would be undetectable by SDS-PAGE analysis. SMT assays contained 12.5 μL of 0.5 mM sinapoylglucose in 100 mM potassium phosphate buffer (pH 7.5), 5 μL of 100 mM potassium phosphate buffer (pH 6.0), 5 μL of 1 M malic acid in potassium phosphate buffer (pH 6.0) and 5 μL of E. coli extract corresponding to 100 μg of protein. Assays were incubated for 14 h at 30° C., stopped by addition of 30 μL of methanol and stored at −70° C. before analysis by HPLC. Sinapoylglucose for use in enzyme assays was purified from the sng1 mutant of Arabidopsis (Lorenzen, et al., 1996). HPLC analysis demonstrated that sinapoylmalate was formed by extracts of cells harboring the pET28A-SNG1 construct when incubated in the presence of sinapoylglucose and malate (FIG. 7). Omission of enzyme, sinapoylglucose, or malate eliminated the production of sinapoylmalate, as did the use of extracts of cells harboring only the pET28A vector. This experiment provides conclusive proof that the SNG1 gene encodes SMT.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### BIBLIOGRAPHY

Altschul, et al., 1990. J. Mol. Biol. 215:403-410.

Altschul, et al., 1997. Nucl. Acids Res. 25:3389-3402.

Ammerer, 1983. Methods in Enzymol. 101:192-201.

An, 1987. Meth. Enzymol. 153:292-305.

Ausubel, et al., 1987. Current Protocols in Molecular Biology, published by Greene Publishing Assoc. and Wiley-Interscience.

Baulcombe, et al., 1987. J. Biol. Chem. 262:13726-13735.

Bech and Breddam, 1989. Carlsberg Res. Commun. 54:165-171.

Becker and Guarente, 1991. Methods in Enzymol. 194:182-186.

Bell-Lelong, et al., 1997. Plant Physiol. 113:729-738.

Benbrook, et al., 1986. In Proceedings Bio Expo 1986, Butterworth, Stoneham, Mass., pp. 27-54.

Bent, et al., 1994. Science 265:1856-1860.

Binding, 1985. Regeneration of Plants, Plant Protoplasts, CRC Press, Boca Raton, Fla., pp. 21-73.

Bishop (Ed.), 1994. Guide to Huge Computers, Academic Press, San Diego, Calif.

Bradford, 1976. Anal. Biochem. 72:248-254.

Bradley, 1992. Plant Physiol. 98:1526-1529.

Brock, 1989. “Biotechnology: A Textbook of Industrial Microbiology,” 2d Ed., Sinauer Associates, Inc, Sunderland, Mass.

Buchman and Berg, 1988. Mol. Cell. Biol. 8:4395-4405.

Burger, 1970. In “Medicinal Chemistry” (A. Burger, ed.), 3rd Edition, Wiley, N.Y.

Callis, et al., 1987. Genes Dev. 1:1183-1200.

Carillo and Lipman, 1988. SIAM J. Applied Math. 48:1073.

Chapple, et al., 1992. Plant Cell 4:1413-1424.

Chrispeels, 1991. Ann. Rev. Plant Phys. Plant Mol. Biol. 42:21-53.

Christensen, 1994. Eur. J. Biochem. 220:149-153.

Coombs, et al., 1998. Proteins, pp. 259-311, 1 plate, R. H. Angeletti, Ed., Academic, San Diego, Calif.

Corner, et al., 1965. Nature 207:634-635.

Daboussi, et al., 1989. Curr. Genet. 15:453-456.

Degan, et al., 1994. Proc. Natl. Acad. Sci. USA 91:8209-8213.

Devereaux, et al., 1984. Nucleic Acids Res. 12:387-395.

Doan and Fincher, 1988. J. Biol. Chem. 263:11106-11110.

Dodson and Wlodawer, 1998. Trends Biochem. Sci. 23:347-352.

Doi, et al., 1980. Agric. Biol. Chem. 44:85-92.

Endrizzi, et al., 1994. Biochemistry 33:11106-11120.

Evans, et al., 1983. Protoplasts Isolation and Culture, Handbook of Plant Cell Culture, Macmillan Publishing Company, NY, pp. 124-176.

Fraley, et al., 1993. Proc. Natl. Acad. Sci. (USA) 80:4803.

Freeling and Walbot (Eds.), 1994. The Maize Handbook, Springer, N.Y.

Freeman, et al., 1984. Plant Cell Physiol. 25:1353.

Frohman et al., 1988. PNAS USA 85:8998.

Fromm, et al., 1985. Proc. Natl. Acad. Sci. (USA) 82:5824.

Gerhardt, et al. (Eds.), 1994. “Manual of Methods for General Bacteriology,” American Society for Microbiology, Washington, D.C.

Ghangas and Steffens, 1993. Proc. Natl. Acad. Sci. USA 90:9911-9915.

Ghangas and Steffens, 1995. Arch. Biochem. Biophys. 316:370-377.

Glägen and Seitz, 1992. Planta 186:582-585.

Glägen, et al, 1992. Phytochemistry 31:1593-1601.

Goldsbrough and Cullis, 1981. Nuc. Acids Res. 9:1301-1309.

Gordon-Kamm, 1990. The Plant Cell 2:603-618.

Gräwe, et al., 1992. Planta 187:236-241.

Gribskov and Devereux (Eds.), 1991. Sequence Analysis Primer, Stockton Press, New York.

Griffin, et al. (Eds.), 1994. Computer Analysis of Sequence Data, Part I, Humana Press, New Jersey.

Grimm, 1925. Z Elektrochemie 31:474-480.

Gross, 1983. Z Naturforsch 38c:519-523.

Hallhorn, et al., 1991. Bio./Technol. 9:1090.

Harborne, et al., 1983. Z Naturforsch 38c:1055-1056.

Haseloff, et al., 1988. Nature 334:585-591.

Hayashi, et al., 1975. J Biol. Chem. 250:5221-5226.

Hayashi, et al., 1973. J Biol. Chem. 248:8366-9369.

Hein, et al., 1990. Methods Enzymol. 183:626-645.

Hess, 1987. Intern Rev. Cytol. 107:367.

Hobson-Frohock, et al., 1977. Br. Poult. Sci. 18:539-541.

Hopp and Seitz, 1987. Planta 170:74-85.

Horsch, et al., 1984. Science 233:496-498.

Horsch, et al., 1985. Science 227:1229-1231.

Jones, et al., 1996. Eur. J. Biochern. 235:574-578.

Kim and Hayashi, 1983. Agric. Biol. Chem. 47:2655-2667.

Kindle, 1990. Proc. NatL. Acad. Sci. (USA) 87:1228.

Klein, et al., 1987. Nature 327:70-73.

Köetter, et al., 1990. Curr. Genet. 18:493-500.

Lerner, 1984. Adv. ImmunoL 36:1.

Leung, et al., 1990. Curr. Genet. 17:409-411.

Lesk (Ed.), 1988. Computational Molecular Biology, Oxford University Press, New York.

Liao, et al., 1992. Biochemistry 31:9796-9812.

Liao and Remington, 1990. J. Biol. Chem. 265:6528-6531.

Lim, et al., 2001. J. Biol. Chem. 276:4344-4349.

Loh, et al., 1989. Science 243:217.

Lorenzen, et al., 1996. Plant Physiol. 112:1625-1630.

Luo, et al., 1988. Plant Mol. Biol. Reporter 6:165.

Marrs, et al., 1995. Nature 375:397-400.

Mehta and Mattoo, 1996. Plant Physiol. 110:875-882.

Mehta, et al., 1996. Plant Physiol. 110:883-892.

Melnikov, et al., 1999. Nucleic Acids Research 27(4):1056-1062.

Meyer, et al., 1996. Cloning of plant genes based on genetic map location. In AH Paterson, ed, Genome Mapping in Plants. Academic Press, New York NY, Landes Bioscience Publishers, Austin, Tex., pp 137-154

Meyer, et al., 1994. Science 264:1452-1455.

Michalczuk and Bandurski, 1982. Biochem. J. 207:273-281.

Mock and Strack, 1993. Phytochemistry 32:575-579.

Murashige and Skoog, 1962. Physiol. Plant 15:473-497.

Napoli, et al., 1990. The Plant Cell 2:279-289.

Neuhaus, et al., 1987. Theor. AppL Genet.75:30.

Nielsen, et al., 1997. Protein Engin. 10:1-6.

Nurmann and Strack, 1979. Z Naturforsch 34c:715-720.

Ohara, et al., 1989. PNAS USA 86:5673.

Parsons, et al., 1997. Proc. Natl. Acad. Sci. USA 84:4161-4165.

Paszkowski, et al., 1984. Embo. J. 3:2717-2722.

Pearson, et al., 1988. Proc. Natl. Acad. Sci. U.S.A. 85:2444-2448.

Pena, et al., 1987. Nature 325:274.

Raikhel, 1992. Plant Phys. 100:1627-1632.

Ramos and Winther, 1996. Eur. J. Biochem. 242:29-35.

Ramos, et al., 1994. J. Biol. Chem. 269:7006-7012.

Rogers and Bendich, 1985. Plant Mol. Biol. 5:69-76.

Rogers, et al., 1987. Meth. in Enzymol. 153:2531-277.

Ruegger, et al., 1999. Plant Physiol 119:101-110.

Rychlik, 1993. In White, B. A., Ed., “Methods in Molecular Biology,” 15:31-39, PCR Protocols: Current Methods and Applications. Humania Press, Inc., totowa, N.J.

Sambrook, et al., 1989. “Molecular cloning.” A laboratory manual, 2d Ed., Cold Spring Harbor Laboratory Press, Plainview, N.Y.

Sarthy, et al., 1987. Appl. Environ. Microb. 53:1996-2000.

Schwartz, et al., 1997. Plant Physiol. 114:161-166.

Sharma and Strack, 1985. Planta 163:563-568.

Sheehy, et al., 1988. Proc. Nat'L Acad. Sci. 85:8805-8809.

Silhavy, et al., 1984. Experiments with Gene Fusions, Cold Spring Harbor Laboratory, Cold Press Spring Harbor, N.Y.

Smith (Ed.), 1993. Biocomputing Informatics and Genome Projects, Academic press, New York.

Sprague and Dudley, Eds., 1988. Corn and Corn Improvement, 3^{rd} Ed., American Society of Agronomy, Madison, Wis.

Strack, 1982. Planta 155:31-36.

Strack, et al., 1983. Z Naturforsch 38c:21-27.

Strack, et al., 1980. Z Naturforsch 35c:963-966.

Strack and Sharma, 1985. Physiol. Plant 65:45-50.

Tabor, et al., 1985. Proc. Acad. Sci. USA 82:1074.

Thein and Wallace, 1986. “The use of oligonucleotide as specific hybridization probes in the Diagnosis of Genetic Disorders,” in Human Genetic Diseases: A Practical Approach, K. E. David, Ed., pp. 33-50, IRL Press, Hemdon, Va.

Tomes, et al., 1995. Direct DNA Transfer into Intact Plant Cells Via Microprojectile Bombardment in PLANT CELL, TISSUE AND ORGAN CULTURE, FUNDAMENTAL METHODS, O. L. Gamborg and G. C. Phillips, Eds., Springer-Verlag Berlin Heidelberg, N.Y. 1995, pp. 197-213.

Valls, et al., 1990. J. Cell. Biol. 111:361-368.

Van Ness and Chen, 199. Nucl. Acids Res.19:5143-5151.

Villegas and Kojima, 1986. J. Biol. Chem. 261:8729-8733.

Vogt, et al., 1993. Arch. Biochem. Biophys. 300:622-628.

von Heinje, 1987. Sequence Analysis in Molecular Biology, Academic Press, New York.

Wajant, et al., 1994. Plant Mol. Biol. 26:735-746.

Walker, et al., 1992. Proc. Natl. Acad. Sci. USA 89:392.

Walker-Simmons and Ryan, 1980. Phytochemistry 19:43-47.

Washio and Ishikawa, 1994. Biochim. Biophys. Acta. 1199:311-314.

Weising, et al., 1988. Ann. Rev. Genet. 22:421-477.

Weissbach and Weissbach, Eds., 1988. Methods for Plant Molecular Biology, Academic Press, Inc., San Diego, Calif.

Wetmur and Davidson, 1968. J. Mol. Biol. 31:349-370.

Whetten, et al., 1998. Annu. Rev. Plant Physiol. Plant Mol. Biol. 49:585-609.

Widmer and Johansen, 1979. Carlsberg Res. Commun. 44:37-46.

Widmer, et al., 1980. Carlsberg Res. Commun. 45:453-463.

Wolf, et al., 1996. FEBS Lett 384:31-34.

Zambrisky, et al., 1983. EMBO J 2:2143-2150.

Zhijian Li, et al., 1992. Plant Physiol. 100:662-668.

Zhou, et al., 1983. Methods in Enzymology 101:433.

What is claimed is:

1. A method for producing transgenic plants comprising:
(i) transforming plant cells with an isolated DNA comprising a nucleic acid or its complement, said nucleic acid comprises a nucleotide sequence selected from the group consisting of:
(a) a nucleotide sequence coding for Arabidopsis SMT comprising an amino acid sequence selected from the group consisting of SEQ ID NO:4 and SEQ ID NO:6;
(b) a nucleotide sequence coding for a protein comprising an amino acid sequence that has at least 90% identity with an amino acid sequence selected from the group consisting of SEQ ID NO:4 and SED ID NO:6;
(c) a nucleotide sequence coding for all or a substantial portion of the amino acid sequence selected from the group consisting of SEQ ID NO:4 and SEQ ID NO:6;
(d) a nucleotide sequence that is substantially similar to an isolated nucleic acid molecule coding for all or a substantial portion of the amino acid sequence selected from the group consisting of SEQ ID NO:4 and SEQ ID NO:6;
(e) a nucleotide sequence that hybridizes with one of the nucleotide sequences of (a)-(d) under the following hybridization conditions: 40% formamide, with 6× SSC, 0.1× SSC, at 55° C. and washed with 2× SSC, 0.1% SDS followed by 0.1× SSC, 0.1% SDS; and
(ii) selecting transformed plant cells containing said DNA, and
(iii) regenerating said transgenic plant from said transformed plant cells.

2. An isolated DNA comprising a nucleic acid or its complement, said nucleic acid comprises a nucleotide sequence coding for a member selected from the group consisting of:
(a) Arabidopsis SMT comprising an amino acid sequence selected from the group selected from SEQ ID NO:4 and SEQ ID NO:6;
(b) a protein comprising an amino acid sequence that has at least 90% identity with an amino acid sequence selected from the group consisting of SEQ ID NO:4 and SEQ ID NO:6; and
(c) all or a substantial portion of the amino acid sequence selected from the group consisting of SEQ ID NO:4 and SEQ ID NO:6.

3. The isolated DNA of claim 2, wherein said nucleic acid comprises a nucleotide sequence selected from the group consisting of:
(a) SEQ ID NO:3;
(b) the complement of SEQ ID NO:3;
(c) SEQ ID NO:5; and
(d) the complement of SEQ ID NO:5.

4. The isolated DNA of claim 2, wherein said nucleic acid comprises a nucleotide sequence or its complement selected from the group consisting of:
(a) a nucleotide sequence that has at least 90% identity with a nucleotide sequence selected from the group consisting of SEQ ID NO:4 and SEQ ID NO:6;
(b) a nucleotide sequence that is substantially similar to an isolated nucleic acid molecule coding for all or a substantial portion of the amino acid sequence selected from the group consisting of SEQ ID NO:4 and SEQ ID NO:6; and
(c) a nucleotide sequence that hybridizes with one of the nucleotide sequences of (a) or (b) under the following hybridization conditions: 40% formamide, with 6× SSC, 0.1× SSC, at 55° C. and washed with 2× SSC, 0.1% SDS followed by 0.1× SSC, 0.1% SDS.

5. An isolated DNA comprising a nucleic acid which comprises an Arabidopsis SMT signal peptide.

6. A DNA molecule comprising a heterologous promoter operably linked to the isolated DNA of claim 2 or a fragment thereof which is capable of altering secondary metabolism

7. The DNA molecule of claim 6, wherein said secondary metabolism is altered by an antisense mechanism.

8. The DNA molecule of claim 6, wherein said secondary metabolism is altered by a sense increase or suppression mechanism.

9. A DNA molecule comprising a heterologous promoter operably linked to the isolated DNA of claim 3 or a fragment thereof which is capable of altering secondary metabolism.

10. A DNA molecule comprising the isolated DNA of claim 5 operably linked to a second, heterologous nucleic acid.

11. A vector comprising the isolated DNA of claim 2.

12. A vector comprising the isolated DNA of claim 3.

13. A vector comprising the isolated DNA of claim 5.

14. A vector comprising the DNA molecule of claim 6.

15. A vector comprising the DNA molecule of claim 10.

16. A transformed plant cell comprising the isolated DNA of claim 2 or a fragment thereof which is capable of altering secondary metabolism.

17. A transformed plant cell comprising the isolated DNA of claim 5.

18. A transformed plant cell comprising the DNA molecule of claim 6.

19. A transformed plant cell comprising the DNA molecule of claim 10.

20. A transformed plant comprising the isolated DNA of claim 2, or fragment thereof which is capable of altering secondary metabolism.

21. A transformed plant comprising the isolated DNA of claim 5.

22. A transformed plant comprising the DNA molecule of claim 6.

23. A transformed plant comprising the DNA molecule of claim 10.

24. An isolated polypeptide comprising an amino acid sequence selected from the group consisting of SEQ ID NO:4 and SEQ ID NO:6.

25. Host cells transformed with a DNA molecule comprising a nucleotide sequence selected from the group consisting of:
(a) a nucleotide sequence coding for a member selected from the group consisting of:
(i) Arabidopsis SMT comprising an amino acid sequence selected from the group selected from SEQ ID NO:4 and SEQ ID NO:6;
(ii) a protein comprising an amino acid sequence that has at least 90% identity with an amino acid sequence selected from the group consisting of SEQ ID NO:4 and SEQ ID NO:6; and
(iii) all or a substantial portion of the amino acid sequence selected from the group consisting of SEQ ID NO:4 and SEQ ID NO:6;
(b) SEQ ID NO:3;
(c) the complement of SEQ ID NO:3;
(d) SEQ ID NO:5;
(e) the complement of SEQ ID NO:5;
(f) a nucleotide sequence that has at least 90% identity with a nucleotide sequence selected from the group consisting of SEQ ID NO:4 and SEQ ID NO:6;
(g) a nucleotide sequence that is substantially similar to an isolated nucleic acid molecule coding for all or a substantial portion of the amino acid sequence selected from the group consisting of SEQ ID NO:4 and SEQ ID NO:6; and
(h) a nucleotide sequence that hybridizes with one of the nucleotide sequences of (a) through (g) under the following hybridization conditions: 40% formamide, with 6× SSC, 0.1× SSC, at 55° C. and washed with 2× SSC, 0.1% SDS followed by 0.1× SSC, 0.1% SDS.

26. A method of producing a polypeptide which comprises:
(i) culturing the host cells of claim 25 under conditions suitable for the production of said polypeptide; and
(ii) recovering said polypeptide.

27. A method for producing transgenic plants comprising transforming plant cells with the DNA molecule of claim 10, selecting transformed plant cells containing said DNA molecule and regenerating said transgenic plant from said transformed plant cells.

28. A method for altering the secondary metabolism of a plant comprising transforming said plant with the DNA of claim 2 operably liked to a heterologous promoter.

29. A method for altering the secondary metabolism of a plant comprising transforming said plant with the DNA of claim 3 operably liked to a heterologous promoter.

30. A method for altering the secondary metabolism of a plant comprising transforming said plant with the DNA of claim 5 operably liked to a heterologous promoter.

31. The method of claim 28, wherein said secondary metabolism of monosaccharide esters is altered.

32. The method of claim 29, wherein said secondary metabolism of monosaccharide esters is altered.

33. The method of claim 30, wherein said secondary metabolism of monosaccharide esters is altered.

34. The method of claim 31, wherein said monosaccharide ester is selected from the group consisting of esters of glucose, ribulose, sylulose, psicose, fructose, sorbose, tagatose, sedoheptulose, ribose, arabinose, xylose, lyxose, allose, altrose, mannose, gulose, idose, galactose, and talose.

35. The method of claim 32, wherein said monosaccharide ester is selected from the group consisting of esters of glucose, ribulose, sylulose, psicose, fructose, sorbose, tagatose, sedoheptulose, ribose, arabinose, xylose, lyxose, allose, altrose, mannose, gulose, idose, galactose, and talose.

36. The method of claim 33, wherein said monosaccharide ester is selected from the group consisting of esters of glucose, ribulose, sylulose, psicose, fructose, sorbose, tagatose, sedoheptulose, ribose, arabinose, xylose, lyxose, allose, altrose, mannose, gulose, idose, galactose, and talose.

37. The method of claim 28, wherein the metabolism of monosaccharide ester conjugates of a substrate is altered.

38. The method of claim 37, wherein said substrate is selected from the group consisting of benzoic acid, o-hydroxybenzoic acid, m-hydroxybenzoic acid, 3,4-dihydroxybenzoic acid, vanillic acid, syringic acid, cinnamic acid, o-coumaric acid, m-coumaric acid, caffeic acid, ferulic acid, 5-hydroxyferulic acid, isoferulic acid, and sinapic acid.

39. A method for altering lignin biosynthesis of a plant comprising transforming said plant with a DNA molecule comprising a heterologous promoter operably linked to a nucleic acid comprising a nucleotide sequence selected from the group consisting of:
(a) a nucleotide sequence coding for a member selected from the group consisting of:
(i) Arabidopsis SMT comprising an amino acid sequence selected from the group selected from SEQ ID NO:4 and SEQ ID NO:6;
(ii) a protein comprising an amino acid sequence that has at least 90% identity with an amino acid sequence selected from the group consisting of SEQ ID NO:4 and SEQ ID NO:6; and
(iv) all or a substantial portion of the amino acid sequence selected from the group consisting of SEQ ID NO:4 and SEQ ID NO:6;
(b) SEQ ID NO:3;
(c) the complement of SEQ ID NO:3;
(d) SEQIDNO:5;
(e) the complement of SEQ ID NO:5;
(f) a nucleotide sequence that has at least 90% identity with a nucleotide sequence selected from the group consisting of SEQ ID NO:4 and SEQ ID NO:6;
(g) a nucleotide sequence that is substantially similar to an isolated nucleic acid molecule coding for all or a substantial portion of the amino acid sequence selected from the group consisting of SEQ ID NO:4 and SEQ ID NO:6; and
(h) a nucleotide sequence that hybridizes with one of the nucleotide sequences of (a) through (g) under the following hybridization conditions: 40% formamide, with 6× SSC, 0.1× SSC, at 55° C. and washed with 2× SSC, 0.1% SDS followed by 0.1× SSC, 0.1% SDS.

40. The method of claim 39, wherein said lignin biosynthesis is altered by altering the metabolism of lignin biosynthetic intermediates.

41. The method of claim 40, wherein said intermediates are selected from the group consisting of monosaccharide esters of cinnamic acid, p-coumaric acid, caffeic acid, ferulic acid, 5-hydroxyferulic acid and sinapic acid.

42. A method of altering sinapoylcholine content of a plant comprising transformation of said plant with a DNA molecule comprising a promoter operably linked to a nucleic acid comprising a nucleotide sequence selected from the group consisting of:
(a) a nucleotide sequence coding for a member selected from the group consisting of:
(i) Arabidopsis SMT comprising an amino acid sequence selected from the group selected from SEQ ID NO:4 and SEQ ID NO:6;
(ii) a protein comprising an amino acid sequence that has at least 90% identity with an amino acid sequence selected from the group consisting of SEQ ID NO:4 and SEQ ID NO:6; and
(v) all or a substantial portion of the amino acid sequence selected from the group consisting of SEQ ID NO:4 and SEQ ID NO:6;
(b) SEQID NO:3;
(c) the complement of SEQ ID NO:3;
(d) SEQ ID NO:5;
(e) the complement of SEQ ID NO:5;
(f) a nucleotide sequence that has at least 90% identity with a nucleotide sequence selected from the group consisting of SEQ ID NO:4 and SEQ ID NO:6;
(g) a nucleotide sequence that is substantially similar to an isolated nucleic acid molecule coding for all or a substantial portion of the amino acid sequence selected from the group consisting of SEQ ID NO:4 and SEQ ID NO:6; and
(h) a nucleotide sequence that hybridizes with one of the nucleotide sequences of (a) through (g) under the following hybridization conditions: 40% formamide, with 6× SSC, 0.1× SSC, at 55° C. and washed with 2× SSC, 0.1% SDS followed by 0.1× SSC, 0.1% SDS.

43. A method for altering pathogen resistance of a plant comprising transformation of said plant with a DNA molecule comprising a heterologous promoter operably linked to a nucleic acid comprising a nucleotide sequence selected from the group consisting of:
(a) a nucleotide sequence coding for a member selected from the group consisting
(i) Arabidopsis SMT comprising an amino acid sequence selected from the group selected from SEQ ID NO:4 and SEQ ID NO:6;
(ii) a protein comprising an amino acid sequence that has at least 90% identity with an amino acid sequence selected from the group consisting of SEQ ID NO:4 and SEQ ID NO:6; and
(vi) all or a substantial portion of the amino acid sequence selected from the group consisting of SEQ ID NO:4 and SEQ ID NO:6;
(b) SEQ ID NO:3;
(c) the complement of SEQ ID NO:3;
(d) SEQID NO:5;
(e) the complement of SEQ ID NO:5;
(f) a nucleotide sequence that has at least 90% identity with a nucleotide sequence selected from the group consisting of SEQ ID NO:4 and SEQ ID NO:6;
(g) a nucleotide sequence that is substantially similar to an isolated nucleic acid molecule coding for all or a substantial portion of the amino acid sequence selected from the group consisting of SEQ ID NO:4 and SEQ ID NO:6; and
(h) a nucleotide sequence that hybridizes with one of the nucleotide sequences of (a) through (g) under the following hybridization conditions: 40% formamide, with 6× SSC, 0.1× SSC, at 55° C. and washed with 2× SSC, 0.1% SDS followed by 0.1× SSC, 0.1% SDS.

44. The method of claim 43, wherein said pathogen is an insect.

45. A method for altering UV-B resistance of a plant comprising transformation of said plant with a DNA molecule comprising a heterologous promoter operably linked to a nucleic acid comprising a nucleotide sequence selected from the group consisting of:
(a) a nucleotide sequence coding for a member selected from the group consisting of:
(i) Arabidopsis SMT comprising an amino acid sequence selected from the group selected from SEQ ID NO:4 and SEQ ID NO:6;
(ii) a protein comprising an amino acid sequence that has at least 90% identity with an amino acid sequence selected from the group consisting of SEQ ID NO:4 and SEQ ID NO:6; and
(vii) all or a substantial portion of the amino acid sequence selected from the group consisting of SEQ ID NO:4 and SEQ ID NO:6;
(b) SEQ ID NO:3;
(c) the complement of SEQ ID NO:3;
(d) SEQID NO:5;
(e) the complement of SEQ ID NO: 5;
(f) a nucleotide sequence that has at least 90% identity with a nucleotide sequence selected from the group consisting of SEQ ID NO:4 and SEQ ID NO:6;
(g) a nucleotide sequence that is substantially similar to an isolated nucleic acid molecule coding for all or a substantial portion of the amino acid sequence selected from the group consisting of SEQ ID NO:4 and SEQ ID NO:6; and
(h) a nucleotide sequence that hybridizes with one of the nucleotide sequences of (a) through (g) under the following hybridization conditions: 40% formamide, with 6× SSC, 0.1× SSC, at 55° C. and washed with 2× SSC, 0.1% SDS followed by 0.1× SSC, 0.1% SDS.


SEQUENCE LISTING (17 sequences)

SEQ ID NO: 1 - DNA, 1518 bp, Arabidopsis thaliana;
aattttataaagatcctatgtctatccgtaaatggactaatctttagacacacagagaatataatgagtttgaaaataaagtttctgcttctgcttgtcttgtatcatcatgttgattctgcctctatcgtcaagtttcttcctggttttgaaggccctcttcctttcgaacttgaaaccgggtacattggtattggtgaggacgagaatgtgcaatttttctactatttcatcaaatctgaaaacaatccaaaagaagatcctcttcttatatggttaaatggaggacctggatgttcttgtcttggtggtattatttttgagaacggaccggtgggtttgaagtttgaggtgttcaacggaagtgctccttctttgttctctactacatattcatggacaaagatggcaaacattatattcttggatcagccagtaggatctggcttctcctactcaaaaactccaattgataaaactggtgacataagtgaagtaaagaggacccatgagtttcttcaaaagtggctaagcaggcatccacaatatttctccaaccctttatatgttgttggagattcttattccggtatgattgtcccggccctcgttcaagaaatctcacaaggaaattatatatgttgcgaacctcctataaatctacagggttatatgcttggaaaccctgtaacatatatggactttgaacaaaacttccgcattccatatgcttatggtatgggattaatctctgacgaaatctatgagccaatgaagagaatctgcaacggaaattattacaatgtggatccatctaacacacaatgtttgaaacttactgaagaatatcataagtgcactgccaaaataaatatccatcacatattaacaccagattgcgatgtaaccaatgtaacatctcctgattgttattattatccatatcatctcattgaatgttgggctaacgacgagagcgttcgcgaagctcttcatattgaaaagggtagtaaaggaaaatgggcgcgatgtaatcggactattccatacaatcacgacattgtaagcagcataccatatcacatgaataacagcatcagtggataccgatctcttatttacagtggtgatcacgacatcgcggtcccttttcttgcaactcaagcctggataagatctctcaattactcccccattcataactggaggccatggatgataaacaatcaaatcgctggatacacgagagcttattccaataagatgacatttgctactatcaaaggaggtggacacacggcagagtatagaccaaacgagacctttatcatgttccaaaggtggatcagtggccaacccctgtaacaaaaggcttatgaccttcacctataattatctaccaactaatatccacgttaagcgcagttgtttgtgttgaaatgtttttgttgtttgctttgttgcattcttttgtgctttatgttacaattttatgtgttttatgtactacagttcatt

SEQ ID NO: 2 - protein, 433 aa, Arabidopsis thaliana
MSLKIKFLLLLVLYHHVDSASIVKFLPGFEGPLPFELETGYIGIGEDENVQFFYYFIKSENNPKEDPLLIWLNGGPGCSCLGGIIFENGPVGLKFEVFNGSAPSLFSTTYSWTKMANIIFLDQPVGSGFSYSKTPIDKTGDISEVKRTHEFLQKWLSRHPQYFSNPLYVVGDSYSGMIVPALVQEISQGNYICCEPPINLQGYMLGNPVTYMDFEQNFRIPYAYGMGLISDEIYEPMKRICNGNYYNVDPSNTQCLKLTEEYHKCTAKINIHHILTPDCDVTNVTSPDCYYYPYHLIECWANDESVREALHIEKGSKGKWARCNRTIPYNHDIVSSIPYHMNNSISGYRSLIYSGDHDIAVPFLATQAWIRSLNYSPIHNWRPWMINNQIAGYTRAYSNKMTFATIKGGGHTAEYRPNETFIMFQRWISGQPL

SEQ ID NO: 3 - DNA, 1302 bp, Arabidopsis thaliana
atgagtttgaaaataaagtttctgcttctgcttgtcttgtatcatcatgttgattctgcctctatcgtcaagtttcttcctggttttgaaggccctcttcctttcgaacttgaaaccgggtacattggtattggtgaggacgagaatgtgcaatttttctactatttcatcaaatctgaaaacaatccaaaagaagatcctcttcttatatggttaaatggaggacctggatgttcttgtcttggtggtattatttttgagaacggaccggtgggtttgaagtttgaggtgttcaacggaagtgctccttctttgttctctactacatattcatggacaaagatggcaaacattatattcttggatcagccagtaggatctggcttctcctactcaaaaactccaattgataaaactggtgacataagtgaagtaaagaggacccatgagtttcttcaaaagtggctaagcaggcatccacaatatttctccaaccctttatatgttgttggagattcttattccggtatgattgtcccggccctcgttcaagaaatctcacaaggaaattatatatgttgcgaacctcctataaatctacagggttatatgcttggaaaccctgtaacatatatggactttgaacaaaacttccgcattccatatgcttatggtatgggattaatctctgacgaaatctatgagccaatgaagagaatctgcaacggaaattattacaatgtggatccatctaacacacaatgtttgaaacttactgaagaatatcataagtgcactgccaaaataaatatccatcacatattaacaccagattgcgatgtaaccaatgtaacatctcctgattgttattattatccatatcatctcattgaatgttgggctaacgacgagagcgttcgcgaagctcttcatattgaaaagggtagtaaaggaaaatgggcgcgatgtaatcggactattccatacaatcacgacattgtaagcagcataccatatcacatgaataacagcatcagtggataccgatctcttatttacagtggtgatcacgacatcgcggtcccttttcttgcaactcaagcctggataagatctctcaattactcccccattcataactggaggccatggatgataaacaatcaaatcgctggatacacgagagcttattccaataagatgacatttgctactatcaaaggaggtggacacacggcagagtatagaccaaacgagacctttatcatgttccaaaggtggatcagtggccaacccctgtaa

SEQ ID NO: 4 - protein, 433 aa, Arabidopsis thaliana; identical to SEQ ID NO: 2

SEQ ID NO: 5 - DNA, 1245 bp, Arabidopsis thaliana
gcctctatcgtcaagtttcttcctggttttgaaggccctcttcctttcgaacttgaaaccgggtacattggtattggtgaggacgagaatgtgcaatttttctactatttcatcaaatctgaaaacaatccaaaagaagatcctcttcttatatggttaaatggaggacctggatgttcttgtcttggtggtattatttttgagaacggaccggtgggtttgaagtttgaggtgttcaacggaagtgctccttctttgttctctactacatattcatggacaaagatggcaaacattatattcttggatcagccagtaggatctggcttctcctactcaaaaactccaattgataaaactggtgacataagtgaagtaaagaggacccatgagtttcttcaaaagtggctaagcaggcatccacaatatttctccaaccctttatatgttgttggagattcttattccggtatgattgtcccggccctcgttcaagaaatctcacaaggaaattatatatgttgcgaacctcctataaatctacagggttatatgcttggaaaccctgtaacatatatggactttgaacaaaacttccgcattccatatgcttatggtatgggattaatctctgacgaaatctatgagccaatgaagagaatctgcaacggaaattattacaatgtggatccatctaacacacaatgtttgaaacttactgaagaatatcataagtgcactgccaaaataaatatccatcacatattaacaccagattgcgatgtaaccaatgtaacatctcctgattgttattattatccatatcatctcattgaatgttgggctaacgacgagagcgttcgcgaagctcttcatattgaaaagggtagtaaaggaaaatgggcgcgatgtaatcggactattccatacaatcacgacattgtaagcagcataccatatcacatgaataacagcatcagtggataccgatctcttatttacagtggtgatcacgacatcgcggtcccttttcttgcaactcaagcctggataagatctctcaattactcccccattcataactggaggccatggatgataaacaatcaaatcgctggatacacgagagcttattccaataagatgacatttgctactatcaaaggaggtggacacacggcagagtatagaccaaacgagacctttatcatgttccaaaggtggatcagtggccaacccctgtaa

SEQ ID NO: 6 - protein, 414 aa, Arabidopsis thaliana
ASIVKFLPGFEGPLPFELETGYIGIGEDENVQFFYYFIKSENNPKEDPLLIWLNGGPGCSCLGGIIFENGPVGLKFEVFNGSAPSLFSTTYSWTKMANIIFLDQPVGSGFSYSKTPIDKTGDISEVKRTHEFLQKWLSRHPQYFSNPLYVVGDSYSGMIVPALVQEISQGNYICCEPPINLQGYMLGNPVTYMDFEQNFRIPYAYGMGLISDEIYEPMKRICNGNYYNVDPSNTQCLKLTEEYHKCTAKINIHHILTPDCDVTNVTSPDCYYYPYHLIECWANDESVREALHIEKGSKGKWARCNRTIPYNHDIVSSIPYHMNNSISGYRSLIYSGDHDIAVPFLATQAWIRSLNYSPIHNWRPWMINNQIAGYTRAYSNKMTFATIKGGGHTAEYRPNETFIMFQRWISGQPL

SEQ ID NO: 7 - DNA, 20 bp, Arabidopsis thaliana
gatgcactcgaaatcagcca

SEQ ID NO: 8 - DNA, 20 bp, Arabidopsis thaliana
gcgcggagtcattacagtta

SEQ ID NO: 9 - DNA, 31 bp, Arabidopsis thaliana
cgggtaccagcaaaacgcatcaaccataaac

SEQ ID NO: 10 - DNA, 19 bp, Arabidopsis thaliana
gagggccgggacaatcata

SEQ ID NO: 11 - protein, 17 aa, Arabidopsis thaliana
ASIVKFLPGFEGPLPFE

SEQ ID NO: 12 - DNA, 28 bp, Arabidopsis thaliana
tcatgacctctatcgtcaagtttcttcc

SEQ ID NO: 13 - DNA, 25 bp, Arabidopsis thaliana
gtcgacttacaggggttggccactg

SEQ ID NO: 14 - protein, 433 aa, Arabidopsis thaliana; identical to SEQ ID NO: 2

SEQ ID NO: 15 - protein, 421 aa, Saccaromyces
KIKDPKILGIDPNVTQYTGYLDVEDEDKHFFFWTFESRNDPAKDPVILWLNGGPGCSSLTGLFFELGPSSIGPDLKPIGNPYSWNSNATVIFLDQPVNVGFSYSGSSGVSNTVAAGKDVYNFLELFFDQFPEYVNKGQDFHIAGESYAGHYIPVFASEILSHKDRNFNLTSVLIGNGLTDPLTQYNYYEPMACGEGGEPSVLPSEECSAMEDSLERCLGLIESCYDSQSVWSCVPATIYCNNAQLAPYQRTGRNVYDIRKDCEGGNLCYPTLQDIDDYLNQDYVKEAVGAEVDHYESCNFDINRNFLFAGDWMKPYHTAVTDLLNQDLPILVYAGDKDFICNWLGNKAWTDVLPWKYDEEFASQKVRNWTASITDEVAGEVKSYKHFTYLRVFNGGHMVPFDVPENALSMVNEWIHGGFSL

SEQ ID NO: 16 - protein, 411 aa, Triticales
VEPSGHAADRIARLPGQPAVDFDMYSGYITVDEGAGRSLFYLLQEAPEDAQPAPLVLWLNGGPGCSSVAYGASEELGAFRVKPAGAGLVLNEYRWNKVANVLFLDSPAGVGFSYTNTSSDIYTSGDNRTAHDSYAFLAKWFERFPHYKYRDFYIAGESYAGHYVPELSQLVHRSKNPVINLKGFMVGNGLIDDYHDYVGTFEFWWNHGIVSDDTYRRLKEACLHDSFIHPSPACDAATDVATAEQGNIDMYSLYTPVCNSYDPCTERYSTAYYNRRDVQMALHANVTGAMNYTWATCSDTINTHWHDAPRSMLPIYRELIAAGLRIWVFSGDTDAVVPLTATRYSIGALGLPTTTSWYPWYDDQEVGGWSQVYKGLTLVSVRGAGHEVPLHRPRQALVLFQYFLQGKPMPG

SEQ ID NO: 17 - protein, 366 aa, Sorghum bicolor
RPLEYAWNKAANILFAESPAGVGFSYSNTSSDLSMGDDKMAQDTYTFLVKWFERFPHYKYREFYIAGESGHFIPQLSQVVYRNRNNSPFINFQGLLVSSGLTNDHEDMIGMFESWWHHGLISDETRDSGLKVCPGTSFMHPTPECTEVWNKALAEQGNINPYTIYTPTCDREPSPYQRRFWAPHGRAAPPPLMLPPYDPCAVFNSINYLNLPEVQTALHANVSGIVEYPWTVCSNTIFDQWGQAADDLLPVYRELIQAGLRVWVYSGDTDSVVPVSSTRRSLAALELPVKTSWYPWYMAPTEREVGGWSVQYEGLTYVSPSGAGHLVPVHRPAQAFLLFKQFLKGEPMPAEEKNDILLPSQKAPFY